(19) United States
(12) Patent Application Publication
(10) Pub. No.: US 2026/0134985 A1
(43) Pub. Date: May 14, 2026

(54) METHODS AND SYSTEMS FOR REGISTERING INTERNAL AND EXTERNAL COORDINATE SYSTEMS FOR SURGICAL GUIDANCE

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Brian William QUIST, Salem, NH (US); Paul Alexander TORRIE, Marblehead, MA (US); Geoffrey Ian KARASIC, Raynham, MA (US); João Pedro DE ALMEIDA BARRETO, Coimbra (PT); Carolina DOS SANTOS RAPOSO, Coimbra (PT)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/442,182

(22) Filed: Jan. 7, 2026

Related U.S. Application Data
(63) Continuation of application No. PCT/US2024/038340, filed on Jul. 17, 2024.
(60) Provisional application No. 63/514,663, filed on Jul. 20, 2023.
(60) Provisional application No. 63/593,391, filed on Oct. 26, 2023.
(60) Provisional application No. 63/593,407, filed on Oct. 26, 2023.

(51) Int. Cl. G16H 40/40; G16H 30/40

(52) U.S. Cl. CPC G16H 40/40; G16H 30/40

(57) ABSTRACT

Some examples are directed to methods, systems, and related tools for registering an internal coordinate system of a surgical site with an external coordinate system of a surgical room.

### RELATED APPLICATIONS

The application is a continuation of and claims the benefit of International Application No. PCT/US2024/038340 filed Jul. 17, 2024. PCT/US2024/038340 claims priority to U.S. Provisional Patent Application No. 63/514,663, entitled “METHODS AND SYSTEMS FOR REGISTERING INTERNAL AND EXTERNAL COORDINATE SYSTEMS FOR SURGICAL NAVIGATION GUIDANCE,” filed Jul. 20, 2023, to U.S. Provisional Patent Application No. 63/593,391, entitled “SURGICAL APPLICATIONS BASED ON SHARING INFORMATION BETWEEN INTRA-AND EXTRA-ARTICULAR REFERENCE SYSTEMS,” filed Oct. 26, 2023, and to U.S. Provisional Patent Application No. 63/593,407, entitled “TRANSFERENCE OF INFORMATION BETWEEN INTRA-AND EXTRA-ARTICULAR COORDINATE SYSTEMS,” filed Oct. 26, 2023. The applications referenced above are incorporated by reference herein in their entirety.

### BACKGROUND

Surgical procedures in sports medicine typically involve repairs to injured articular tissue and/or bone, and may involve the insertion of implants such as grafts, anchors, and/or other devices.

For example, femoroacetabular impingement (FAI) resulting from damage to the labrum or articular cartilage of the hip resulting from either a bony overgrowth on the neck of the femur (called a cam deformity), a bony overgrowth around the acetabular rim (called a pincer deformity), or a combination of the two, may be treated using a mechanical resection device to remove bone and create an anatomical profile that does not result in impingement during typical ranges of motion. Treatment may be performed with respect to the cam deformity, the pincer deformity, or both. As another example, injury to the anterior cruciate ligament (ACL), which serves as the primary mechanical restraint in the knee to resist anterior translation of the tibia relative to the femur, may be treated with a reconstruction of the ACL. Reconstruction may consist of placement of a substitute graft (e.g., autograft from either the central third of the patellar tendon or the hamstring tendons). The ends of the graft may be placed into respective tunnels prepared through the femur and the tibia and these ends may be attached using interference screws or a suspensory fixation device.

As with other kinds of surgical procedures, to facilitate good patient outcomes and surgical room operational efficiency, it can be desirable for surgical procedures in sports medicine to be minimally invasive. For example, rather than conducting an ACL reconstruction or an FAI treatment by making one or more large incisions to completely expose a surgical site to the direct view of a surgeon, it is common for the surgeon to make one or more small incisions-known as working portals-that are just large enough to enable insertion, from outside of the patient through to the surgical site, of distal ends of instruments to be used by the surgeon. The surgeon may then conduct the procedure by manipulating proximal ends of the instruments from outside of the patient thereby to cause the distal ends of the instruments to operate within the surgical site. Once the procedure inside the joint is completed, the instruments may be withdrawn, and the working portals closed. Due to their small size, the working portals once closed may tend to heal quickly and with little complication.

In a minimally invasive procedure known as arthroscopic surgery, the surgeon may be guided by what is captured within the field of view of an endoscope inserted through a working portal into the surgical site. The procedure may be computer-assisted in the sense that a controller is used for arthroscopic navigation within the surgical site. The controller may provide computer-assistance by tracking locations of various objects within the surgical site, such as the location of a bone and various instruments within an internal frame defined by the three-dimensional coordinate space of the view of the endoscope. Examples of methods and systems for such internal surgical navigation are described in PCT Publication No. WO/2023/034194 to Quist et al. (“Quist”).

Alternatively, the surgeon may be guided during a minimally invasive procedure by what is captured within the field of view of an external camera system within the surgical room. In such a case, the procedure may be computer-assisted in the sense that a controller is used for navigation within the surgical room. The controller may provide computer-assistance by tracking locations of various objects in the surgical room, such as the location of the bone and various instruments within an external frame defined by the three-dimensional coordinate space of the view of the external camera system. Examples of methods and systems for such external surgical navigation are described in PCT Publication No. WO/2022/006041 to Netravali et al. (“Netravali”).

For an arthroscopic procedure, the inside of the surgical site cannot typically be “seen” by an external camera system, and a surgical room cannot typically be “seen” by an endoscope within the surgical site. Given this, procedures conducted by a surgeon as internal-frame, micro-scale procedures are generally independent of those conducted as external-frame, macro-scale procedures, such that any particular computer assistance provided with respect to one of the frames/scales is not applicable to the other. Practically-speaking, however, for a given procedure, a surgeon is required to effectively execute both on the external/macro-scale and on the internal/micro-scale to, for example, make working portals and thereafter execute the procedure internally. For example, positioning of tools, whether for tunnel placement or for bone removal, is mechanically constrained by the placement of the respective working portals.

### SUMMARY

One example is a method for registering internal and external coordinate systems of a surgical system. The method may comprise providing a tool having a distal portion and a proximal portion; capturing, while the proximal portion has a known pose with respect to the distal portion and while the distal portion is at a location within a surgical site and the proximal portion is outside of the surgical site, a pair of images comprising: a first image using a first image-capture system and containing the proximal portion and an external bone fiducial; and a second image using a second image-capture system and containing the distal portion and an internal bone fiducial; processing the pair of images to: determine an external pose of the proximal portion in an external coordinate system of the external bone fiducial; and determine an internal pose of the distal portion in an internal coordinate system of the internal bone fiducial; and generating a spatial transformation between the external and internal coordinate systems based at least on the known pose.

The method may further comprise capturing internal video frames of at least one object and the internal bone fiducial using the second image-capture system; based on the spatial transformation, processing the internal video frames to determine a pose of the at least one object in the external coordinate system; and displaying, on a display device in association with external video frames captured using the first image-capture system, indicia representing the pose of the at least one object in the external coordinate system.

The method may further comprise capturing external video frames of at least one object and the external bone fiducial using the first image-capture system; based on the spatial transformation, processing the external video frames to determine a pose of the at least one object in the internal coordinate system; and displaying, on a display device in association with internal video frames captured using the second image-capture system, indicia representing the pose of the at least one object in the internal coordinate system.

The method may further comprise forming the pair of images by simultaneously capturing the first image and the second image.

In the method, the forming may be conducted based at least on a known offset between a time of image capture by the first image-capture system and a time of image capture by the second image-capture system.

In the method, the forming may be conducted based at least on a known rate of image capture by the first image-capture system and a known rate of image-capture by the second image-capture system.

In the method, the second image-capture system may comprise at least one of: an endoscopic camera and a needle scope.

In the method, the first image-capture system may comprise at least one of: a visual camera and an infrared camera.

In the method, the first image may include an external tool fiducial associated with the proximal portion, and wherein processing the pair of images to determine the external pose of the proximal portion in the external coordinate system of the external bone fiducial may comprise: processing the first image to determine the external pose of the external tool fiducial in the external coordinate system of the external bone fiducial.

In the method, the second image may include an internal tool fiducial associated with the distal portion, and wherein processing the pair of images to determine the internal pose of the distal portion in the internal coordinate system of the internal bone fiducial may comprise processing the second image to determine the internal pose of the internal tool fiducial in the internal coordinate system of the internal bone fiducial.

Yet another example is a surgical system. The surgical system may comprise a tool comprising a distal portion and a proximal portion, the proximal portion having a known pose with respect to the distal portion, the distal portion dimensioned to be received within a surgical site while the proximal portion is outside of the surgical site; a first image-capture system outside of the surgical site; a second image-capture system inside the surgical site; processing structure comprising at least one computer processor, the processing structure in communication with the first image-capture system and the second image-capture system and configured for: capturing, while the distal portion is at a location within the surgical site and the proximal portion is outside of the surgical site, a pair of images comprising: a first image using the first image-capture system and containing the proximal portion and an external bone fiducial; and a second image using the second image-capture system and containing the distal portion and an internal bone fiducial; processing the pair of images to: determine an external pose of the proximal portion in an external coordinate system of the external bone fiducial; and determine an internal pose of the distal portion in an internal coordinate system of the internal bone fiducial; and generating a spatial transformation between the external and internal coordinate systems based at least on the known pose.

In the example surgical system, the processing structure may be configured for: capturing internal video frames of at least one object and the internal bone fiducial using the second image-capture system; based on the spatial transformation, processing the internal video frames to determine a pose of the at least one object in the external coordinate system; and displaying, on a display device in association with external video frames captured using the first image-capture system, indicia representing the pose of the at least one object in the external coordinate system.

In the example surgical system, the processing structure may be configured for: capturing external video frames of at least one object and the external bone fiducial using the first image-capture system; based on the spatial transformation, processing the external video frames to determine a pose of the at least one object in the internal coordinate system; and displaying, on a display device in association with internal video frames captured using the second image-capture system, indicia representing the pose of the at least one object in the internal coordinate system.

In the example surgical system, the processing structure may be configured for: forming the pair of images by simultaneously capturing the first image and the second image.

In the example surgical system, the processing structure may be configured for conducting the forming based at least on a known offset between a time of image capture by the first image-capture system and a time of image capture by the second image-capture system.

In the example surgical system, the processing structure may be configured for conducting the forming based at least on a known rate of image capture by the first image-capture system and a known rate of image-capture by the second image-capture system.

In the example surgical system, the second image-capture system may comprise at least one of: an endoscopic camera and a needle scope.

In the example surgical system, the first image-capture system may comprise at least one of: a visual camera and an infrared camera.

In the example surgical system, the first image may include an external tool fiducial associated with the proximal portion, and wherein processing the pair of images to determine the external pose of the proximal portion in the external coordinate system of the external bone fiducial may comprise: processing the first image to determine the external pose of the external tool fiducial in the external coordinate system of the external bone fiducial.

In the example surgical system, the second image may include an internal tool fiducial associated with the distal portion, and wherein processing the pair of images to determine the internal pose of the distal portion in the internal coordinate system of the internal bone fiducial may comprises processing the second image to determine the internal pose of the internal tool fiducial in the internal coordinate system of the internal bone fiducial.

Yet another example is a method for registering internal and external coordinate systems of a surgical system. The method may comprise: providing a tool having a distal portion and a proximal portion; capturing, for each of at least three locations within a surgical site, while the proximal portion has a fixed pose with respect to the distal portion and while the distal portion is at the location and the proximal portion is outside of the surgical site, a pair of images comprising: a first image using a first image-capture system and containing the proximal portion and an external bone fiducial; and a second image using a second image-capture system and containing the distal portion and an internal bone fiducial; processing each pair of images to: determine an external pose of the proximal portion in an external coordinate system of the external bone fiducial; and determine an internal pose of the distal portion in an internal coordinate system of the internal bone fiducial; and generating a spatial transformation between the external and internal coordinate systems based at least on the external poses and the internal poses.

The example method may further comprise capturing video frames of at least one object and the internal bone fiducial using the second image-capture system; based on the spatial transformation, processing the video frames to determine a pose of the at least one object in the external coordinate system; and displaying, on a display device in association with video frames captured using the first image-capture system, indicia representing the pose of the at least one object in the external coordinate system.

The example method may further comprise capturing video frames of at least one object and the external bone fiducial using the first image-capture system; based on the spatial transformation, processing the video frames to determine a pose of the at least one object in the internal coordinate system; and displaying, on a display device in association with video frames captured using the second image-capture system, indicia representing the pose of the at least one object in the internal coordinate system.

The example method may further comprise forming each pair by pairing those of the at least one first and second images that were captured simultaneously.

In the example method, the forming may be conducted based on a known offset between a time of image capture by the first image-capture system and a time of image capture by the second image-capture system.

In the example method, the forming may be conducted based at least on a known rate of image capture by the first image-capture system and a known rate of image capture by the second image-capture system.

In the example method, generating the spatial transformation between the external and internal coordinate systems may comprise registering each of the locations in the external coordinate system to a 3D bone model thereby to generate a first bone model transformation; registering each of the locations in the internal coordinate system to the 3D bone model thereby to generate a second bone model transformation; and generating the spatial transformation between the external and internal coordinate systems based at least on the first bone model transformation and the second bone model transformation.

In the example method, the second image-capture system may comprise at least one of: an endoscopic camera and a needle scope.

In the example method, the first image-capture system may comprise at least one of: a visual camera and an infrared camera.

In the example method, each first image may include an external tool fiducial associated with the proximal portion, and wherein processing each pair of images to determine each external pose of the proximal portion in the external coordinate system of the external bone fiducial may comprises processing each first image to determine each external pose of the external tool fiducial in the external coordinate system of the external bone fiducial.

In the example method, each second image may include an internal tool fiducial associated with the distal portion, and wherein processing each pair of images to determine each internal pose of the distal portion in the internal coordinate system of the internal bone fiducial may comprise processing each second image to determine each internal pose of the internal tool fiducial in the internal coordinate system of the internal bone fiducial.

Yet another example is a surgical system. The surgical system may comprise a tool comprising a distal portion and a proximal portion, the proximal portion having a fixed pose with respect to the distal portion, the distal portion dimensioned to be received within a surgical site while the proximal portion is outside of the surgical site; a first image-capture system outside of the surgical site; a second image-capture system inside the surgical site; and processing structure comprising at least one computer processor, the processing structure in communication with the first image-capture system and the second image-capture system and configured for: capturing, for each of at least three locations within the surgical site, while the distal portion of the tool is at the location and the proximal portion of the tool is outside of the surgical site, a pair of images comprising: a first image using the first image-capture system and containing the proximal portion and an external bone fiducial; and a second image using the second image-capture system and containing the distal portion and an internal bone fiducial; and processing each pair of images to: determine an external pose of the proximal portion in an external coordinate system of the external bone fiducial; and determine an internal pose of the distal portion in an internal coordinate system of the internal bone fiducial; the processing structure further configured for: generating a spatial transformation between the external and internal coordinate systems based at least on the external poses and the internal poses.

In the example surgical system, the processing structure may be configured for capturing video frames of at least one object and the internal bone fiducial using the second image-capture system; based on the spatial transformation, processing the video frames to determine a pose of the at least one object in the external coordinate system; and displaying, on a display device in association with video frames captured using the first image-capture system, indicia representing the pose of the at least one object in the external coordinate system.

In the example surgical system, the processing structure may be configured for capturing video frames of at least one object and the external bone fiducial using the first image-capture system; based on the spatial transformation, processing the video frames to determine a pose of the at least one object in the internal coordinate system; and displaying, on a display device in association with video frames captured using the second image-capture system, indicia representing the pose of the at least one object in the internal coordinate system.

In the example surgical system, the processing structure may be configured for forming each pair by pairing those of the at least one first and second images that were captured simultaneously.

In the example surgical system, the forming may be conducted based on a known offset between a time of image capture by the first image-capture system and a time of image capture by the second image-capture system.

In the example surgical system, the forming may be conducted based at least on a known rate of image capture by the first image-capture system and a known rate of image capture by the second image-capture system.

In the example surgical system, for generating the spatial transformation between the external and internal coordinate systems the processing structure may be configured for: registering each of the locations in the external coordinate system to a 3D bone model thereby to generate a first bone model transformation; registering each of the locations in the internal coordinate system to the 3D bone model thereby to generate a second bone model transformation; and generating the spatial transformation between the external and internal coordinate systems based at least on the first bone model transformation and the second bone model transformation.

In the example surgical system, the second image-capture system may comprise at least one of: an endoscopic camera and a needle scope.

In the example surgical system, the first image-capture system may comprise at least one of: a visual camera and an infrared camera.

In the example surgical system, each first image may include an external tool fiducial associated with the proximal portion, and wherein for processing each pair of images to determine the external pose of the proximal portion in the external coordinate system of the external bone fiducial the processing structure may be configured for: processing each first image to determine the external pose of the external tool fiducial in the external coordinate system of the external bone fiducial.

In the example surgical system, each second image may include an internal tool fiducial associated with the distal portion, and wherein for processing each pair of images to determine the internal pose of the distal portion in the internal coordinate system of the internal bone fiducial the processing structure may be configured for: processing each second image to determine the internal pose of the internal tool fiducial in the internal coordinate system of the internal bone fiducial.

Yet another example is a method of surgical navigation. The method may comprise: positioning a distal portion of a tool within a surgical site while a proximal portion of the tool is outside of the surgical site, the proximal portion having a known pose with respect to the distal portion; simultaneously capturing: first video frames using a first image-capture system having an external coordinate system, the first video frames including the proximal portion; and second video frames using a second image-capture system, the second video frames including the distal portion, an internal bone fiducial having an internal coordinate system, and an internal object; and during the capturing: for each of a plurality of pairs of the first video frames and the second video frames captured simultaneously: processing the first video frame in the pair to determine a current external pose of the proximal portion in the external coordinate system; processing the second video frame in the pair to: determine a first current internal pose of the distal portion in the internal coordinate system; determine a second current internal pose of the internal bone fiducial in the internal coordinate system; and determine a third current internal pose of the internal object in the internal coordinate system; calculating a pose of the internal object in the external coordinate system based on the current external pose, the first current internal pose, the second current internal pose, the third current internal pose, and the known pose; and displaying, on a display device in association with the first video frames, indicia representing the pose of the internal object in the external coordinate system.

In the example method, the second image-capture system may comprise at least one of: an endoscopic camera and a needle scope.

In the example method, the first image-capture system may comprise at least one of: a visual camera and an infrared camera.

In the example method, the visual camera and the display device may be components of a head-mounted-display (HMD) device.

In the example method, processing the first video frame to determine a current external pose of the proximal portion in the external coordinate system may comprise: processing the first video frame to determine the external pose of an external tool fiducial that is associated with the proximal portion.

In the example method, processing the second video frame to determine a current internal pose of the distal portion in the internal coordinate system may comprise: processing the second video frame to determine the internal pose of an internal tool fiducial that is associated with the distal portion.

Yet another example is a surgical system. The example surgical system may comprise: a tool comprising a distal portion and a proximal portion, the proximal portion having a known pose with respect to the distal portion, the distal portion dimensioned to be received within a surgical site while the proximal portion is outside of the surgical site; a first image-capture system outside of the surgical site and having an external coordinate system; a second image-capture system inside the surgical site; and processing structure comprising at least one computer processor, the processing structure in communication with the first image-capture system and the second image-capture system and configured for: simultaneously capturing: first video frames using the first image-capture system, the first video frames including the proximal portion; and second video frames using the second image-capture system, the second video frames including the distal portion, an internal bone fiducial having an internal coordinate system, and an internal object; and during the capturing: for each of a plurality of pairs of the first video frames and the second video frames captured simultaneously: processing the first video frame in the pair to determine a current external pose of the proximal portion in the external coordinate system; processing the second video frame in the pair to: determine a first current internal pose of the distal portion in the internal coordinate system; determine a second current internal pose of the internal bone fiducial in the internal coordinate system; and determine a third current internal pose of the internal object in the internal coordinate system; calculating a pose of the internal object in the external coordinate system based on the current external pose, the first current internal pose, the second current internal pose, the third current internal pose, and the known pose; and displaying, on a display device in association with the first video frames, indicia representing the pose of the internal object in the external coordinate system.

In the example surgical system, the second image-capture system may comprise at least one of: an endoscopic camera and a needle scope.

In the example surgical system, the first image-capture system may comprise at least one of: a visual camera and an infrared camera.

In the example surgical system, the visual camera and the display device may be components of a head-mounted-display (HMD) device.

In the example surgical system, the processing structure may be configured for processing the first video frame to determine a current external pose of the proximal portion in the external coordinate system including: processing the first video frame to determine the external pose of an external tool fiducial that is associated with the proximal portion.

In the example surgical system, the processing structure may be configured for processing the second video frame to determine a current internal pose of the distal portion in the internal coordinate system including: processing the second video frame to determine the internal pose of an internal tool fiducial that is associated with the distal portion.

### BRIEF DESCRIPTION OF THE DRAWINGS

For a detailed description of example embodiments, reference will now be made to the accompanying drawings in which:

FIG. 1 shows an anterior or front elevation view of right knee, with the patella removed;

FIG. 2 shows a posterior or back elevation view of the right knee;

FIG. 3 shows a view of the femur from below and looking into the intercondylar notch;

FIG. 4 shows a surgical system in accordance with at least some embodiments;

FIG. 5 shows a conceptual drawing of a surgical site with various objects within the surgical site being tracked, in accordance with at least some embodiments;

FIG. 6 shows a conceptual drawing of a surgical room with various objects within the surgical room being tracked, in accordance with at least some embodiments;

FIG. 7 is an example video display showing portions of a femur and having visible therein a bone fiducial, in accordance with at least some embodiments;

FIG. 8 shows a tool for use during merging of internal and external coordinate systems, in accordance with at least some embodiments;

FIG. 9 shows an example of components of a surgical system arrangement for merging the internal and external coordinate systems, in accordance with at least some embodiments;

FIG. 10 shows an example position and orientation of the tool within the surgical system arrangement for capturing pairs of 3D points in the internal and external coordinate systems;

FIG. 10A shows an arthroscopic view of portions of the surgical system arrangement of FIG. 10;

FIG. 11 shows another example position and orientation of the tool within the surgical system arrangement for capturing pairs of 3D points in the internal and external coordinate systems;

FIG. 12 shows another example position and orientation of the tool within the surgical system arrangement for capturing pairs of 3D points in the internal and external coordinate systems;

FIG. 13 is a conceptual drawing showing the relationship between the internal and external coordinate systems being defined by a stored mathematical transformation generated using the 3D points;

FIG. 14 shows a conceptual drawing of a surgical room with various objects within the surgical room being tracked, in accordance with at least some embodiments;

FIG. 15 shows a display of a surgical system including an arthroscopic view of a surgical site as in FIG. 14 and a first indicia for surgical guidance positioned and oriented based on a first position and orientation of objects being tracked within the surgical room, in accordance with at least some embodiments;

FIG. 16 shows a display of a surgical system including an arthroscopic view of a surgical site as in FIG. 14 and the first indicia positioned and oriented based on a second position and orientation of objects being tracked within the surgical room, in accordance with at least some embodiments;

FIG. 17 shows a display of a surgical system including an arthroscopic view of a surgical site as in FIG. 14 and the first indicia positioned and oriented based on a third position and orientation of objects being tracked within the surgical room, in accordance with at least some embodiments;

FIG. 18 shows a display of a surgical system including an arthroscopic view of a surgical site as in FIG. 14 and each of the first indicia and a second indicia for surgical guidance positioned and oriented based on a fourth position and orientation of objects being tracked within the surgical room, in accordance with at least some embodiments;

FIG. 19 shows a display of a surgical system including an arthroscopic view of a surgical site as in FIG. 14 and each of the first indicia and the second indicia positioned and oriented based on a fifth position and orientation of objects being tracked within the surgical room, in accordance with at least some embodiments;

FIG. 20 shows a display of a surgical system including an arthroscopic view of a surgical site as in FIG. 14 and each of the first indicia and the second indicia positioned and oriented based on a sixth position and orientation of objects being tracked within the surgical room, in accordance with at least some embodiments;

FIG. 21 shows a display of a surgical system including an arthroscopic view of a surgical site as in FIG. 14 and each of the first indicia and the second indicia positioned and oriented based on a seventh position and orientation of objects being tracked within the surgical room, in accordance with at least some embodiments;

FIG. 22 shows an example of components of a surgical system arrangement for merging internal and external coordinate systems with a set of transformations that may be combined to produce a spatial transformation between external and internal coordinate systems;

FIG. 23 shows another example of components of a surgical system arrangement for merging internal and external coordinate systems with a set of transformations that may be combined to produce a spatial transformation between the external and internal coordinate systems;

FIG. 24A shows example components of a surgical system in which a user is wearing a head mounted display (HMD) having a camera and capturing an external bone fiducial and portions of the exterior of a patient's anatomy;

FIG. 24B shows contents of a display of the HMD of FIG. 24A;

FIG. 25A shows example components of a surgical system in which a user is wearing a head mounted display (HMD) having a camera and capturing an external tool fiducial and portions of the exterior of a patient's anatomy;

FIG. 25B shows contents of a display of the HMD of FIG. 25A;

FIG. 26 shows a method of registering internal and external coordinate systems of a surgical system, in accordance with at least some embodiments;

FIG. 27 shows another method of registering internal and external coordinate systems of a surgical system, in accordance with at least some embodiments;

FIG. 28 shows a method of surgical navigation, in accordance with at least some embodiments; and

FIG. 29 shows a computer system in accordance with at least some embodiments.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

“Receiving . . . a . . . location” shall mean receiving data indicative of location on a bone within a coordinate space (e.g., a coordinate space of a view of an endoscope). Thus, example systems and methods may “receive . . . a revised-tunnel entry location” being data indicative of a proposed location of a tunnel entry point within a three-dimensional coordinate space. Other example systems and methods may “receive . . . a plurality of locations on a bone” being data indicative locations of an outer surface of a bone as part of registering a bone to a three-dimensional bone model.

An endoscope having “a single optical path” through an endoscope shall mean that the endoscope is not a stereoscopic endoscope having two distinct optical paths separated by an interocular distance at the light collecting end of the endoscope. The fact that an endoscope has two or more optical members (e.g., glass rods, optical fibers) forming a single optical path shall not obviate the status as a single optical path.

“Throughbore” shall mean an aperture or passageway through an underlying device. However, the term “throughbore” shall not be read to imply any method of creation. Thus, a throughbore may be created in any suitable way, such as drilling, boring, laser drilling, or casting.

“Counterbore” shall mean an aperture or passageway into an underlying device. In cases in which the counterbore intersects another aperture (e.g., a throughbore), the counterbore may thus define an internal shoulder. However, the term “counterbore” shall not be read to imply any method of creation. A counterbore may be created in any suitable way, such as drilling, boring, laser drilling, or casting.

“Processing structure” shall mean a single processing device, processor, microprocessing device, microprocessor, computing device, computer, computer system or other device that, like these, can be instructed to and/or configured to conduct computational processing, or an arrangement of multiple processing devices, processors, microprocessing devices, microprocessors, computing devices, computers, computer systems and/or other devices that, like these, can be instructed to and/or configured to conduct computational processing.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various examples are directed to a tool for merging, or registering, an external coordinate system of a surgical room containing a patient with an internal coordinate system of a surgical site within the patient.

Various examples are directed to methods and systems for generating a transform for merging, or registering, the external coordinate system with the internal coordinate system.

Various examples are directed to methods and systems for providing surgical guidance in the internal coordinate system context based on tracking of instruments in the external coordinate system context and the transform. Furthermore, various examples are directed to methods and systems for providing surgical guidance in the external coordinate system context based on tracking of instruments in the internal coordinate system context and the transform.

The discussion below features various examples developed in the context of ACL repair. However, the techniques are applicable to many types of surgical procedures involving an external coordinate system for a surgical room containing a patient and an internal coordinate system of a surgical site within the patient. Such example surgical procedures may include other types of ligament repair, such as medial collateral ligament repair, lateral collateral ligament repair, and posterior cruciate ligament repair. Other examples of surgical procedures may include FAI treatment or other procedures involving resection. Moreover, the various example methods and systems can also be used for planning and placing anchors to reattach soft tissue, such as reattaching the labrum of the hip, the shoulder, or the meniscal root. Furthermore, the various example methods and systems can also be used for planning and navigation of instruments with respect to an anatomy. Thus, the description and developmental context shall not be read as a limitation of the applicability of the teachings. In order to orient the reader, the specification first turns a description of the knee.

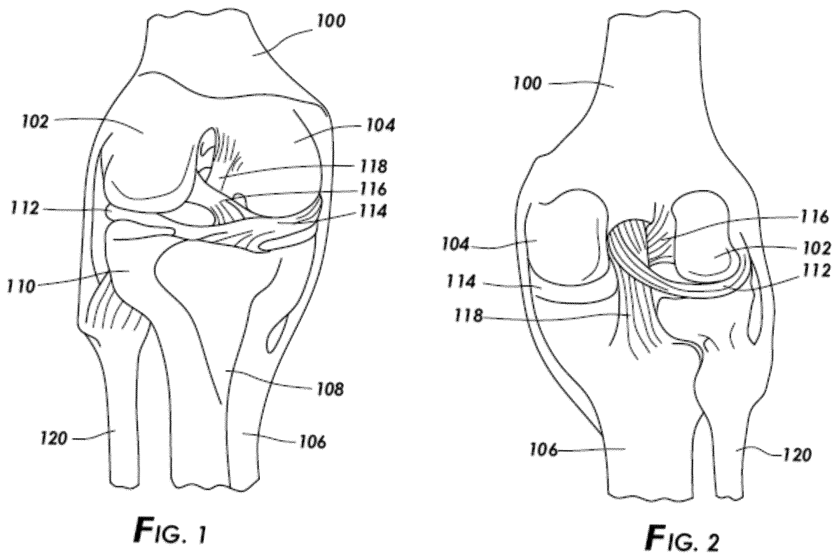

FIG. 1 shows an anterior or front elevation view of a right knee, with the patella removed. In particular, visible in FIG. 1 is lower portion of the femur 100 including the outer or lateral condyle 102 and the inner or medial condyle 104. The femur 100 and condyles 102 and 104 are in operational relationship to a tibia 106 including the tibial tuberosity 108 and Gerdy's tubercle 110. Disposed between the femoral condyles 102 and 104 and the tibia 106 are the lateral meniscus 112 and the medial meniscus 114. Several ligaments are also visible in the view of FIG. 1, such as the ACL 116 extending from the lateral side of femoral notch to the medial side of the tibia 106. Oppositely, the posterior cruciate ligament 118 extends from medial side of the femoral notch to the tibia 106. Also visible is the fibula 120, and several additional ligaments that are not specifically numbered.

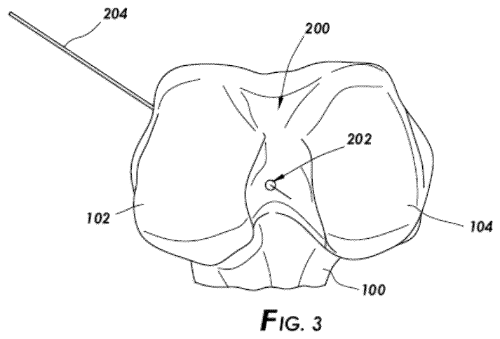

FIG. 2 shows a posterior or back elevation view of the right knee. In particular, visible in FIG. 2 is lower portion of the femur 100 including the lateral condyle 102 and the medial condyle 104. The femur 100 and femoral condyles 102 and 104 again are in operational relationship to the tibia 106, and disposed between the femoral condyles 102 and 104 and the tibia 106 are the lateral meniscus 112 and the medial meniscus 114. FIG. 2 further shows the ACL 116 extending from the lateral side of femoral notch to the medial side of the tibia 106, though the attachment point to the tibia 106 is not visible. The posterior cruciate ligament 118 extends from medial side of the femoral notch to the tibia 106, though the attachment point to the femur 100 not visible. Again, several additional ligaments are shown that are not specifically numbered.

The most frequent ACL injury is a complete tear of the ligament. Treatment involves reconstruction of the ACL by placement of a substitute graft (e.g., autograft from either the patellar tendon, quad tendon, or the hamstring tendons). The graft is placed into tunnels prepared within the femur 100 and the tibia 106. The current standard of care for ACL repair is to locate the tunnels such that the tunnel entry point for the graft is at the anatomical attachment location of the native ACL. Such tunnel placement at the attachment location of the native ACL attempts to recreate original knee kinematics. In arthroscopic surgery, the location of the tunnel through the tibia 106 is relatively easy to reach, particularly when the knee is bent or in flexion. However, the tunnel through the femur 100 resides within the intercondylar notch. Depending upon the physical size of the patient and the surgeon's selection for location of the port through the skin, and through which the various instruments are inserted into the knee, it may be difficult to reach the attachment location of the native ACL to the femur 100.

FIG. 3 shows a view of the femur from below and looking into the intercondylar notch. In particular, visible in FIG. 3 are the lateral condyle 102 and the medial condyle 104. Defined between the femoral condyles 102 and 104 is the femoral notch 200. The femoral tunnel may define an inside aperture 202 within the femoral notch 200, the inside aperture 202 located on wall of the lateral condyle 102 and displaced into the posterior portion of the femoral notch 200. The femoral tunnel extends through the femur 100 and forms an outside aperture on the outside or lateral surface of the femur 100 (the outside aperture not visible in FIG. 3). FIG. 3 shows an example drill wire 204 that may be used to create an initial tunnel or pilot hole. Once the surgeon verifies that the pilot hole is closely aligned with a planned-tunnel path, the femoral tunnel is created by boring or reaming with another instrument (e.g., a reamer) that may use the drill wire 204 as a guide. In some cases, a socket or counter-bore is created on the intercondylar notch side to accommodate the width of the graft that extends into the bone, and that counterbore may also be created using another instrument (e.g., reamer) that may use the drill wire 204 as a guide.

Drilling of a tunnel may take place from either direction. Considering the femoral tunnel again as an example, the tunnel may be drilled from the outside or lateral portion of the femur 100 toward and into the femoral notch 200, which is referred to as an “outside-in” procedure. Oppositely, the example femoral tunnel may be drilled from the inside of the femoral notch 200 toward and to the lateral portion of the femur 100, which is referred as an “inside-out” procedure. The various examples discussed below are equally applicable to outside-in or inside-out procedures. Outside-in procedures may additionally use a device which holds the drill wire on the outside portion, and physically shows the expected tunnel location of the inside aperture within the knee. However, the device for the outside-in procedure is difficult to use in arthroscopic procedures, and thus many arthroscopic repairs use the inside-out procedure. The further examples discussed below are thus based on an inside-out procedure, but such should not be read as a limitation. The specification now turns to an example surgical system.

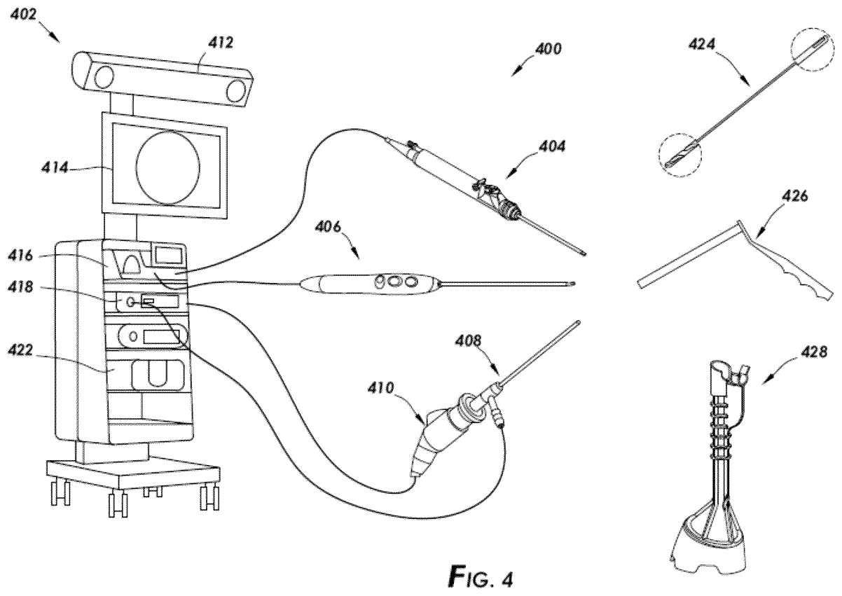

FIG. 4 shows a surgical system (not to scale) in accordance with at least some embodiments. In particular, the example surgical system 400 comprises a tower or device cart 402, an example mechanical resection instrument 404, an example plasma-based ablation instrument (hereafter just ablation instrument 406), and an endoscope in the example form of an arthroscope 408 and attached camera head 410. The endoscope 408 defines a light connection or light post 420 to which light is provided, and the light is routed internally within the endoscope 408 to illuminate a surgical field at the distal end of the endoscope 408. The device cart 402 may comprise a camera 412 (illustratively shown as a stereoscopic camera), a display device 414, a resection controller 416, and a camera control unit (CCU) together with an endoscopic light source and video controller 418. In example cases the CCU and video controller 418 provides light to the light post 420 of the arthroscope 408, displays images received from the camera head 410. In example cases, the CCU and video controller 418 also implements various additional aspects, such as calibration of the arthroscope and camera head, displaying planned-tunnel paths on the display device 414, receiving revised-tunnel entry locations, calculating revised-tunnel paths, and calculating and displaying various parameters that show the relationship between the revised-tunnel path and the planned-tunnel path. Thus, the CCU and video controller is hereafter referred to as surgical controller 418. In other cases, however, the CCU and video controller may be a separate and distinct system from the controller that handles aspects of intraoperative changes, yet the separate devices would nevertheless be operationally coupled.

The example device cart 402 further includes a pump controller 422 (e.g., single or dual peristaltic pump). Fluidic connections of the mechanical resection instrument 404 and ablation instrument 406 are not shown so as not to unduly complicate the figure. Similarly, fluidic connections between the pump controller 422 and the patient are not shown so as not to unduly complicate the figure. In the example system, both the mechanical resection instrument 404 and the ablation instrument 406 are coupled to the resection controller 416 being a dual-function controller. In other cases, however, there may be a mechanical resection controller separate and distinct from an ablation controller. The example devices and controllers associated with the device cart 402 are merely examples, and other examples include vacuum pumps, patient-positioning systems, robotic arms holding various instruments, ultrasonic cutting devices and related controllers, patient-positioning controllers, and robotic surgical systems.

FIG. 4 further shows additional instruments that may be present during an example ACL repair. In particular, FIG. 4 shows an example guide wire or drill wire 424 of a drill (not shown in FIG. 4) and an aimer 426. The drill wire 424 may be used to create an initial or pilot tunnel through the bone. In some cases, the diameter of the drill wire may be about 2.4 millimeters (mm), but larger and smaller diameters for the drill wire 424 may be used. The example drill wire 424 is shown with magnified portions on each end, one to show the cutting elements on the distal end of the drill wire 424, and another magnified portion to show a connector for coupling to chuck of a drill. Once the surgeon drills the pilot tunnel, the surgeon and/or the surgical controller 418 (discussed more below) may then assess whether the pilot tunnel matches or closes matches the planned-tunnel path. If the pilot tunnel is deemed sufficient, then the drill wire 424 may be used as a guide for creating the full-diameter throughbore for the tunnel, and possibly also for creating a counterbore associated with intercondylar notch to accommodate the graft. While in some cases the drill wire alone may be used when creating the pilot tunnel, in yet still other cases the surgeon may use the aimer 426 to help guide and place the drill wire 424 at the designed tunnel-entry location.

FIG. 4 also shows that the example system may comprise a calibration assembly 428. As explained in further detail in PCT Publication No. WO/2023/034194 to Quist et al. (“Quist”), the calibration assembly 428 may be used to detect optical distortion in images received by the surgical controller 418 through the arthroscope 408 and attached camera head 410. Additional tools and instruments will be present, such as a drill (not shown in FIG. 4) for drilling with the drill wire 424, various reamers for creating the throughbore and counterbore aspects of the tunnel, and various tools for suturing and anchoring the graft in place. These additional tools and instruments are not shown so as not to further complicate the figure.

The specification now turns to a workflow for an example ACL repair. The workflow may be conceptually divided into planning and repair. The repair workflow may be further conceptually divided into optical system calibration, model registration, tunnel-path planning, working portal creation, tunnel creation, and tunnel placement analysis. Each will be addressed in turn.

### Planning

In accordance with various examples, an ACL repair starts with imaging (e.g., X-ray imaging, computed tomography (CT), magnetic resonance imaging (MRI)) of the knee of the patient, including the relevant anatomy like the lower portion of the femur, the upper portion of the tibia, and the articular cartilage. The discussion that follows assumes MRI imaging, but again many different types of imaging may be used. The MRI imaging can be segmented from the image slices such that a volumetric model or three-dimensional model of the anatomy is created. Any suitable currently available, or after developed, segmentation technology may be used to create the three-dimensional model. More specifically to the example of ACL repair and specifically selecting a tunnel path through the femur, a three-dimensional bone model of the lower portion of the femur, including the femoral condyles, is created.

Using the three-dimensional bone model, an operative plan is created that comprises choosing a planned-tunnel path through the femur, including locations of the apertures of the bone that define the ends of the tunnel. For an example inside-out repair, the aperture within the femoral notch is the entry location for the drilling, and the aperture on the lateral surface of the femur is the exit location. For an outside-in repair, the entry and exit locations for drilling are swapped. Still assuming an inside-out repair, the entry location may be selected to be the same as, or close to, the attachment location of the native ACL to the femur within the femoral notch. In some cases, selecting the entry location within the femoral notch may involve use of a Bernard & Hertel Quadrant or grid placed on a fluoroscopic image, or placing the Bernard & Hertel Quadrant on a simulated fluoroscopic image created from the three-dimensional bone model. Based on use of the Bernard & Hertel Quadrant, an entry location for the tunnel is selected. For an inside-out repair, selection of the exit location is less restrictive, not only because the portion of the tunnel proximate to the exit location is used for placement of the anchor for the graft, but also because the exit location is approximately centered in the femur (considered anteriorly to posteriorly), and thus issues of bone wall thickness at the exit location are of less concern. In some cases, a three-dimensional bone model of the proximal end of the tibia is also created, and the surgeon may likewise choose planned-tunnel path(s) through the tibia.

The results of the planning may comprise: a three-dimensional bone model of the distal end of the femur; a three-dimensional bone model for a proximal end of the tibia; an entry location and exit location through the femur and thus a planned-tunnel path for the femur; and an entry location and exit location through the tibia and thus a planned-tunnel path through the tibia. Other surgical parameters may also be selected during the planning, such as tunnel throughbore diameters, tunnel counterbore diameters and depth, desired post-repair flexion, and the like, but those additional surgical parameters are omitted so as not to unduly complication the specification.

### Repair

The specification now turns to repair aspects. The repair aspects include steps and procedures for setting up the surgical system to perform the various repairs. It is noted, however, that some of the repair aspects (e.g., optical system calibration), may take place before any working portals (known also as ports or incisions) are made through the patient's skin, and in fact before the patient is wheeled into the surgical room. Nevertheless, such steps and procedures may be considered repair as they take place in the surgical setting and with the surgical equipment and instruments used to perform the actual repair.

The example ACL repair is conducted arthroscopically and is computer-assisted in the sense the surgical controller 418 is used for arthroscopic navigation within the surgical site. More particularly, in example systems the surgical controller 418 provides computer-assistance during the ligament repair by tracking location of various objects within the surgical site, such as the location of the bone within the internal three-dimensional coordinate space of the view of the arthroscope, and location of the various instruments (e.g., the drill wire 424, the aimer 426) within the internal three-dimensional coordinate space of the view of the arthroscope. Furthermore, in example systems the surgical controller 418 provides computer-assistance during the ligament repair by tracking location of the bone within the external three-dimensional coordinate space of the view of the camera 412 and location of the various instruments within the external three-dimensional coordinate space of the view of the camera 412. The specification turns to brief description of such tracking techniques.

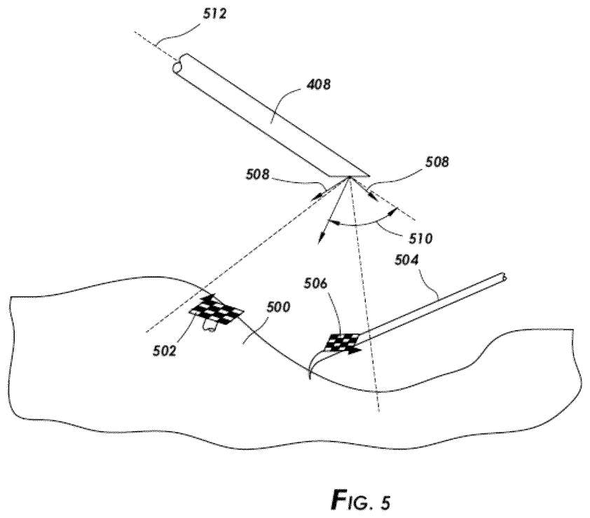

FIG. 5 shows a conceptual drawing of a surgical site with various objects within the surgical site. In particular, visible in FIG. 5 is a distal end of the arthroscope 408, a portion of a bone 500 (e.g., femur), a bone fiducial 502 within the surgical site, a touch probe 504, and a probe fiducial 506. Each is addressed in turn.

The distal end of the arthroscope 408 is designed and constructed to illuminate the surgical site with visible light received by way of the light post (not shown). In the example of FIG. 5, the illumination is illustrated by arrows 508. The illumination provided to the surgical site is reflected by various objects and tissues within the surgical site, and the reflected light that returns to the distal end enters the arthroscope 408, propagates along an optical channel within the arthroscope 408, and is eventually incident upon a capture array within the camera head 410 (FIG. 4). The images detected by the capture array within the camera head 410 are sent electronically to the surgical controller 418 (FIG. 4) and displayed on the display device 414 (FIG. 4). In accordance with example systems, the arthroscope 408 has a single optical path through the arthroscope for capturing images of the surgical site, notwithstanding that the single optical path may be constructed of two or more optical members (e.g., glass rods, optical fibers). That is to say, in example systems and methods the computer-assisted navigation provided by the arthroscope 408, camera head 410, and surgical controller 418 is provided with the arthroscope 408 that is not a stereoscopic endoscope having two distinct optical paths separated by an interocular distance at the distal end of the endoscope.

During a surgical procedure, a surgeon selects an arthroscope with a viewing direction beneficial for the planned surgical procedure. Viewing direction refers to a line residing at the center of an angle subtended by the outside edges or peripheral edges of the view of an endoscope. The viewing direction for some arthroscopes is aligned with the longitudinal central axis of the arthroscope, and such arthroscopes are referred to as “zero degree” arthroscopes (e.g., the angle between the viewing direction and the longitudinal central axis of the arthroscope is zero degrees). The viewing direction of other arthroscopes forms a non-zero angle with the longitudinal central axis of the arthroscope. For example, for a 30° arthroscope the viewing direction forms a 30° angle to the longitudinal central axis of the arthroscope, the angle measured as an obtuse angle beyond the distal end of the arthroscope. In many cases for ACL repair, the surgeon selects a 30° arthroscope or a 45° arthroscope based on location the port created through the skin of the patient. In the example of FIG. 5, the view angle 510 of the arthroscope 408 forms a non-zero angle to the longitudinal central axis 512 of the arthroscope 408.

Still referring to FIG. 5, within the view of the arthroscope 408 is a portion of the bone 500, along with the bone fiducial 502, the touch probe 504, and the probe fiducial 506. The bone fiducial 502 is shown as a planar element having a pattern disposed thereon, though other shapes for the bone fiducial 502 may be used (e.g., a square block with a pattern on each face of the block). The bone fiducial 502 may be attached to the bone 500 in any suitable form (e.g., a fastener, such as a screw). The pattern of the bone fiducial is designed to provide information regarding the orientation of the bone fiducial 502 in the internal three-dimensional coordinate space of the view of the arthroscope 408. More particularly, the pattern is selected such that the orientation of the bone fiducial 502, and thus the orientation of the underlying bone 500, may be determined from images captured by the arthroscope 408 and attached camera head 410 (FIG. 4).

The probe fiducial 506 is shown as a planar element attached to the touch probe 504. The touch probe 504 may be used, as discussed more below, to “paint” the surface of the bone 500 as part of the registration of the bone 500 to the three-dimensional bone model, and the touch probe 504 may also be used to indicate revised-tunnel entry locations in the case of changes to the tunnel paths to be made after initial planning. The probe fiducial 506 is shown as a planar element having a pattern disposed thereon, though other shapes for the probe fiducial 506 may be used (e.g., a square block surrounding the touch probe 504 with a pattern on each face of the block). The pattern of the probe fiducial 506 is designed to provide information regarding the pose (i.e., orientation and position; 6 degrees of freedom) of the probe fiducial 506 in the internal three-dimensional coordinate space of the view of the arthroscope 408. More particularly, the pattern is selected such that the orientation of the probe fiducial 506, and thus the location of the tip of the touch probe 504, may be determined from images captured by the arthroscope 408 and attached camera head 410 (FIG. 4).

Other instruments within the view of the arthroscope 408 may also have fiducials, such as the drill wire 424 (FIG. 4), the drill, and aimer 426 (FIG. 4), but the additional instruments are not shown so as not unduly complicate the figure.

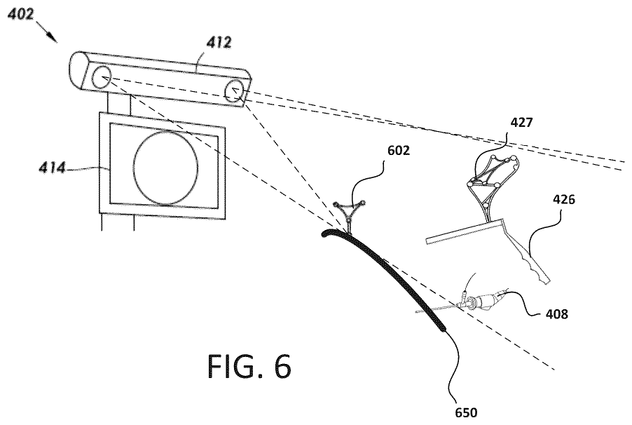

In addition to, or in place of, tracking location based on the view through the arthroscope 408, the location of the distal end of one or more of the instruments may be tracked by other methods and systems. For example, for devices that rigidly extend out of the surgical site (e.g., the drill), the location may be tracked by an optical array coupled to the aimer and viewed through the camera 412 such as a stereoscopic camera. FIG. 6 shows a conceptual drawing of a surgical room with various objects within the surgical room, with the patient anatomy 650 delineating the surgical site internal to the patient from the surgical room external to the patient. In particular, visible to camera 412 in FIG. 6 is a proximate end of an arthroscope 408, a bone fiducial 602, an aimer 426, and an aimer fiducial 427.

The images captured by the arthroscope 408 and attached camera head 410 are subject to optical distortion in many forms. For example, the visual field between distal end of the arthroscope 408 and the bone 500 within the surgical site is filled with fluid, such as bodily fluids and saline used to distend the joint. Many arthroscopes have one or more lenses at the distal end that widen the field of view, and creating wider field of view causes a “fish eye” effect in the captured images. Further, the optical elements within the arthroscope (e.g., rod lenses) may have optical aberrations inherent to the manufacturing and/or assembly process. Further still, the camera head 410 may have various optical elements for focusing the images receives onto the capture array, and the various optical elements may have aberrations inherent to the manufacturing and/or assembly process. As explained in further detail in Quist, in example systems and methods, prior to use within each surgical procedure, the endoscopic optical system is calibrated to account for the various optical distortions. In an example calibration procedure, the example surgical controller 418 creates a characterization function that characterizes optical distortion between the calibration target and the capture array within the camera head 410. The characterization function may include a calibration for determining orientation of fiducial markers visible within the surgical site (e.g., bone fiducial 502, probe fiducial 506) by way of the arthroscope 408 and attached camera head 410.

The next example step in the repair procedure is the registration of the bone model(s). That is, during the planning stage, imaging (e.g., MRI) of the knee takes place, including the relevant anatomy like the lower portion of the femur, the upper portion of the tibia, and the articular cartilage. The imaging can be segmented such that a volumetric model or three-dimensional model of the anatomy is created from cross-sectional images captured during the imaging. More specifically to the example of ACL repair, and specifically selecting a tunnel path through the femur, a three-dimensional bone model of the lower portion of the femur is created during the planning.

During the repair, the three-dimensional bone models are provided to the surgical controller 418. Again using the example of ACL repair, and specifically computer-assisted navigation for tunnel paths through the femur, the three-dimensional bone model of the lower portion of the femur is provided to the surgical controller 418. Thus, the surgical controller 418 receives the three-dimensional bone model, and assuming the arthroscope 408 is inserted into the knee by way of a port through the patient's skin, the surgical controller 418 also receives video images of the femur.

In order to relate the three-dimensional bone model to the images received by way of the arthroscope 408 and camera head 410, the surgical controller 418 registers the three-dimensional bone model to the images of the femur received by way of the arthroscope 408 and camera head 410.

In accordance with example methods, a fiducial marker or bone fiducial (e.g., bone fiducial 502 of FIG. 5) is attached to the femur. The bone fiducial placement is such that the bone fiducial is within the field of view of the arthroscope 408, but in a location spaced apart from the expected tunnel entry/exit point through the lateral condyle. More particularly, in example cases the bone fiducial is placed within the intercondylar notch superior to or above the expected location of the tunnel through lateral condyle.

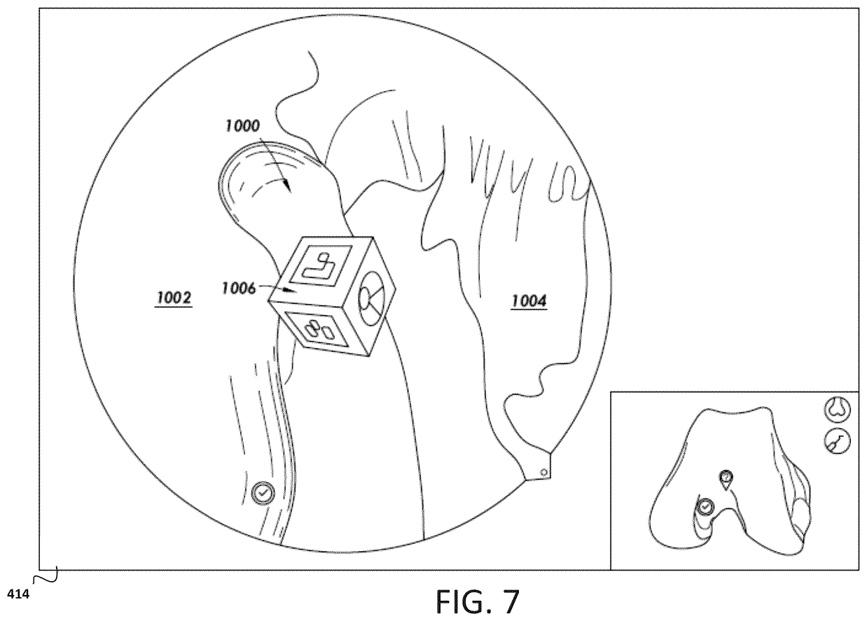

FIG. 7 is an example video display showing portions of a femur and a bone fiducial. The display may be shown, for example, on the display device 414 (FIG. 4) associated with the device cart 402 (FIG. 4), or any other suitable location. In particular, visible in FIG. 7 is a femoral notch or intercondylar notch 1000, a portion of the lateral condyle 1002, a portion the medial condyle 1004, and an example bone fiducial 1006. The bone fiducial 1006 is a fiducial comprising a cube member. Of the six outer faces of the cube member, the bottom face is associated with an attachment feature (e.g., a screw). The bottom face will be close to or will abut the bone when the bone fiducial 1006 is secured in place, and thus will not be visible in the view of the arthroscope 408 (FIG. 4). The outer face opposite the bottom face includes a placement feature used to hold the bone fiducial 1006 prior to placement, and to attach the bone fiducial 1006 to the underlying bone. Of the remaining four outer faces of the cube member (only two of the remaining faces are visible), each of the four outer faces has a machine-readable pattern thereon, and in some cases each machine-readable pattern is unique. Once placed, the bone fiducial 1006 represents a fixed location on the outer surface of the bone in the view of the arthroscope 408, even as the position of the arthroscope 408 is moved and changed relative to the bone fiducial 1006. Initially, the location of the bone fiducial 1006 with respect to the three-dimensional bone model is not known to the surgical controller 418, hence the need for the registration of the three-dimensional bone model.

In order to relate or register the bone visible in the video images to the three-dimensional bone model, the surgical controller 418 (FIG. 4) is provided and thus receives a plurality of locations of an outer surface of the bone. For example, the surgeon may touch a plurality of locations using the touch probe 504 (FIG. 5). As previously discussed, the touch probe 504 comprises a probe fiducial 506 (FIG. 5) visible in the video images captured by the arthroscope 408 (FIG. 4) and camera head 410 (FIG. 4). The physical relationship between the distal end of the touch probe 504 and the probe fiducial 506 is known by the surgical controller 418, and thus as the surgeon touches each of the plurality of locations on the outer surface of the bone, the surgical controller 418 gains an additional “known” location of the outer surface of the bone relative to the bone fiducial 1006. Given that the touch probe 504 is an inflexible instrument, in other examples the tracking of the touch probe 504 may be by optical tracking of an optically-reflective array outside the surgical site (e.g., tracking by the camera 412 (FIG. 4)) yet attached to the portion of the touch probe 504 inside the surgical site.

In some cases, particularly when portions of the outer surface of the bone are exposed to view, receiving the plurality of locations of the outer surface of the bone may involve the surgeon “painting” the outer surface of the bone. “Painting” is a term of art that does not involve application of color or pigment, but instead implies motion of the touch probe 504 when the distal end of the touch probe 504 is touching bone.

Further details of registering a three-dimensional bone model to images of a bone received by way of the arthroscope 408 and camera head 410 will not be described further herein. However, a number of systems, methods and procedures for conducting such registration are described in Quist.

Using the three-dimensional bone model an operative plan may be created that comprises a planned-tunnel path through the bone, including locations of the apertures into the bone that define the ends of the tunnel. In some cases, however, the surgeon may elect not to use planned-tunnel path, and thus elect not use the planned entry location, exit location, or both. Such an election can be based any of a number of reasons. For example, intraoperatively the surgeon may not be able to access the entry location for the planned-tunnel path, and thus may need to move the entry location to ensure sufficient access. As another example, during the repair procedure the surgeon may determine that the planned tunnel entry location is misaligned with the attachment location of the native ACL to the femur. Further still, during the repair procedure the surgeon may determine the tunnel entry location is too close to the posterior wall of the femur, increasing the likelihood of a bone chip sometimes referred to as a “back wall blowout.” Regardless of the reason for the election to change the tunnel path, in example systems the surgical controller 418 may enable the surgeon to intraoperatively select a revised-tunnel entry, a revised-tunnel exit (if needed), and thus a revised-tunnel path through the bone.

A number of systems, methods and procedures for intraoperatively selecting a revised-tunnel entry, a revised-tunnel exit, and thus a revised-tunnel path through the bone are described in Quist.

The next example step in the repair procedure is the merging of internal and external coordinate spaces. The location within the external three-dimensional coordinate space of the camera 412 may be transformed into the internal three-dimensional coordinate space of the view of the example arthroscope to determine location of the distal end of aimer 426 within the surgical site. The linkage between coordinate spaces using a transformation may be useful to provide a surgeon with surgical guidance. For example, a surgeon may be provided with an indication, on a display corresponding to the view of the arthroscope 408 within the surgical site, as to where the distal end of aimer 426 is currently positioned with respect to the view of the arthroscope 408. It may be appreciated that, at times, aimer 426 may be positioned with respect to arthroscope 408 such that no part of aimer 426 or any internal fiducial relating to the aimer 426 or drill wire 424 is within the field of view of arthroscope 408. It may nevertheless be of value to a surgeon to be provided with guidance, when observing the arthroscopic view on, for example, display 414, about the current location and orientation of the distal end of aimer 426 with respect to the arthroscopic view. Knowledge as to the current location and orientation of the distal end of aimer 426 with respect to the arthroscopic view may equip the surgeon with guidance as to which changes in orientation and/or position of the arthroscope 408 and/or of the aimer 426 could bring the distal end of the aimer 426 into the field of view of arthroscope. Accordingly, by tracking the proximal end of the aimer 426 using the position of fiducial 427 with respect to bone fiducial 602 in the field of view of camera 412, and then transforming the position and orientation of the proximal end of the aimer 426 from this external coordinate system to the internal coordinate system of the surgical site, a representation of the position and orientation of the distal end of the aimer 426 may be usefully provided in conjunction with the arthroscopic view.

As another example in which a linkage between coordinate spaces using a transformation may be useful to provide a surgeon with surgical guidance, a surgeon may be provided with an indication, on a display corresponding to the view of the camera 412 in the external three-dimensional coordinate system or of another system such as a head-mounted display worn by a surgeon and itself having a related external three-dimensional coordinate system, as to where a working portal could be created on the patient that would be proximal to, and usefully positioned with respect to, an entry point and trajectory of a planned tunnel. It will be appreciated that, by linking the internal and external coordinate systems, a position at the exterior of patient—on the surface of the patient's skin, for example—could be related to the orientation and position of the bone tunnel to be drilled. For example, a visual indicia may be provided in the view of the camera 412 or other external camera that corresponds to an extension through and beyond the bone tunnel itself, in one or both tunnel directions, towards the exterior of the patient. The extension as represented by a position and orientation of a line in the internal coordinate system of the surgical site corresponding to the planned bone tunnel could be transformed to a line having a respective position and orientation in the external coordinate system. Such a transformed line may then be represented in the external coordinate system and, where such a transformed line is deemed to intersect with location(s) on the exterior of the patient in the external coordinate system context, a visual indicia representing one or more useful working portal location(s) may be provided to the surgeon in conjunction with the view of camera 412 and/or in conjunction with the view of a head-mounted camera or some other external camera.

In examples provided, a tool that provides a fixed or fixable physical relationship between an arthroscopically-viewable uniquely machine-recognizable feature on or at a distal portion of the tool and an externally-viewable uniquely machine-recognizable feature on or at a proximal portion of the tool, may be useful in methods of linking or merging internal and external three dimensional coordinate systems. In one example, during the coordinate system merging process, the relative pose between a machine-recognizable aspect at the distal portion of the tool and a machine-recognizable aspect at the proximal portion of the tool is fixed, though not necessarily known. That is, the two machine-recognizable aspects cannot change pose with respect to each other. In another example, during the coordinate system merging process, the relative pose between the machine-recognizable aspect at the distal portion of the tool and a machine-recognizable aspect at the proximal portion of the tool is fixed and is also known. That is, the two machine-recognizable aspects both cannot change pose with respect to each other and their relative pose is known such that data about this known relative pose is usable by the surgical controller during the coordinate system merging process. In some examples, such uniquely machine-recognizable aspects may each be machine-readable fiducials attached to or otherwise associated with respective ones of the distal and proximal portions of the tool. In some examples, such uniquely machine-recognizable features may each be shapes and/or markings on distal and proximal portions of the tools that are recognizable by the surgical controller. The uniquely machine-recognizable feature at the distal portion of a tool may only be reliably discernable arthroscopically, by being of a size and configuration that may be captured within the field of view of an arthroscopic camera, which is configured to capture within its field of view only smaller-scale objects within a surgical site. On the other hand, the uniquely machine-recognizable feature at the proximal portion of the tool may only be reliably discernable using an external camera such as camera 412, configured to capture within its field of view larger-scale objects in a surgical room. In examples, where the uniquely machine-recognizable features are fiducials (i.e., machine-readable fiducials), the fiducial or fiducials at the distal portion is/are physically much smaller than the fiducial or fiducials at the proximal portion, such that the distal fiducial(s) can be fully captured within the field of view of the arthroscope and such that the proximal fiducial(s) can be reliably distinguished from other objects in the surgical room in the view of the external camera while the distal fiducial(s) can be reliably distinguished from other objects in the field of view of the arthroscope.

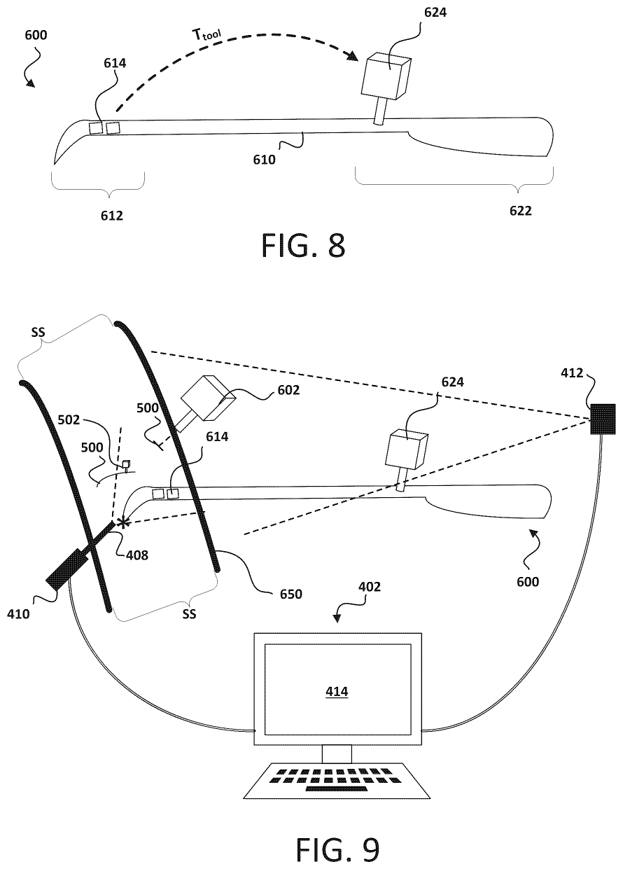

FIG. 8 shows an example of a tool 600 for use in merging internal and external coordinate systems. Tool 600 includes an elongate rigid body 610 having a distal portion 612 and a proximal portion 622. Associated with distal portion 612 is a first fiducial set 614 having, in this example, two redundant fiducials. Associated with proximal portion 622 is a second fiducial set 624 having, in this example, one fiducial. In this example, fiducial set 614 has a fixed physical relationship-a distance and orientation, or “pose”—with fiducial set 624, established by rigid body 610 and their respective poses with respect to rigid body 610. In this example, this fixed pose is known, and the known pose is characterized by a transformation Tₜₒₒₗ between fiducial set 614 and fiducial set 624 that may be determined as one or more calibrations during manufacture of tool 600 or at some other time prior to the registration process described herein. Tₜₒₒₗ itself may be useful for a coordinate system merging process, as described herein. However, the fact that the pose between fiducial set 614 and fiducial set 624 is fixed during a coordinate system merging process can be useful even if, in other examples, the transformation Tₜₒₒₗ itself is not known i.e. not available to surgical controller 418, as also described herein.

Distal portion 612 and, accordingly, fiducial set 614, is dimensioned and configured to be inserted into a surgical site within a patient to be within the field of view of the arthroscopic camera, while proximal portion 622 and, accordingly, fiducial set 624, is dimensioned and configured to remain outside of the surgical site to be within the field of view of the external camera 412. Tool 600 provides a fixed physical relationship between distal portion 612 and proximal portion 622, and thus a fixed physical relationship between second fiducial set 614 and first fiducial set 624. If Tₜₒₒₗ is known, then a known pose between the machine-recognizable features of distal portion 612 and proximal portion 622 is available and can be made use of by surgical controller 418 for a coordinate system merging process.

Alternative forms of tools may provide for different relative poses of a proximal portion with respect to a distal portion. Therefore, if making use of a tool that offers such different relative poses for registration of the internal and external coordinate systems, the proximal portion of such a tool should be arranged to have a fixed pose with respect to the distal portion that does not vary—i.e., is constant-during the coordinate system registration process itself. In some examples, even if the different relative poses are available, it may be possible to obtain a Tₜₒₒₗ for each of the different relative poses.

FIG. 9 shows an example of components of a surgical system arrangement for merging the internal and external coordinate systems. A surgical controller 418 with display 414 is in communication with both an arthroscope including an arthroscope 408 and camera head 410 and with an external camera 412. The field of view of arthroscope 408 within a surgical site SS captures the distal portion 612 of tool 600 including fiducial set 614 along with an internal bone fiducial 502. The field of view of external camera 412 captures the proximal portion 622 of tool 600 including fiducial set 624 and external bone fiducial 602.

In this example, during use of tool 600, this fixed physical relationship between fiducial sets 614 and 624 is not known by the surgical controller 418 (i.e. Tₜₒₒₗ is not available to surgical controller 418) for the process of coordinate system merging that would enable a three-dimensional coordinate in an internal coordinate system indicated by fiducial set 614 as viewed by the arthroscopic camera when tool 600 is held in a fixed position to be paired with a corresponding three-dimensional coordinate in an external coordinate system indicated by fiducial set 624 as viewed by the external camera. Therefore, in this example, during coordinate system merging, multiple pairs of internal and external coordinates may be captured by holding the position of tool 600, and accordingly fiducial sets 614, 624, in different random positions/locations during a merging process. While tool 600 is at each of the positions/locations, the respective internal and external coordinates of a tip of tool 600 (shown with an asterisk, or “*” in FIG. 9) corresponding respectively to fiducial sets 614, 624 within the field of view of respective cameras may be captured simultaneously, and stored in association with each other within surgical controller 418. After at least three different tool positions, the multiple internal coordinates come to represent an internal three-dimensional point cloud of the tip of tool 600, and the multiple external coordinates with which they have been associated come to represent an external three-dimensional point cloud of the tip of tool 600. With the capture of internal and external point clouds, a mathematical transformation may be calculated between the two point clouds thereby to register them and calculate a mathematical transformation between the internal and external coordinate systems.

In examples, the nature of the transformation may be such that the internal coordinate system is established as the global coordinate system, and the external coordinate system is related, through the transformation, to the global coordinate system. In other examples, the nature of the transformation may be such that the external coordinate system is established as the global coordinate system, and the internal coordinate system is related, through the transformation, to the global coordinate system.

In another example, rather than registering an internally-framed point cloud directly to an externally-framed point cloud to calculate the mathematical transformation between internal and external coordinate systems, registrations of each of the two point clouds first to a common 3D bone model that was generated during planning, as described herein, may be conducted. That is, each of the locations in the external coordinate system (for example, the locations of the tip of tool 600 in the frame of the external coordinate system) may be registered to the 3D bone model thereby to generate a first bone model transformation. Similarly, each of the locations in the internal coordinate system (that is, the locations of the tip of tool 600 in the frame of the internal coordinate system) may be registered to the 3D bone model thereby to generate a second bone model transformation. This may be done by using tool 600 to “paint” locations using its tip within the surgical site as described herein thereby to generate multiple locations in each of the external and internal coordinate systems that can, with a sufficient number of locations in each coordinate system, each be mapped to the 3D bone model. With the first and second bone model transformations having been generated, the spatial transformation between the external and internal coordinate systems may be generated based at least on the first bone model transformation and the second bone model transformation. For example, by generating a transformation between the first bone model transformation and the second bone model transformation themselves.

It will be appreciated that other form factors of tools for merging the coordinate systems may be used. For example, distal and proximal fiducial sets on an instrument such as a drill, an ablation device, a resection device, or some other instrument, may be provided and may be used to generate the transformation. Furthermore, it is generally only during the capture of images for a given coordinate system merging that the physical relationship—the relative pose-between the fiducial set 614 and the fiducial set 624 must be fixed and kept constant. That is, while a given example of a tool may be equipped to have variable relative poses between the fiducial set 614 and the fiducial set 624, the relative pose of such a tool should not be made or allowed to vary during a given coordinate system merging procedure so that the physical relationships between all pairs of 3D coordinates is fixed throughout the procedure.

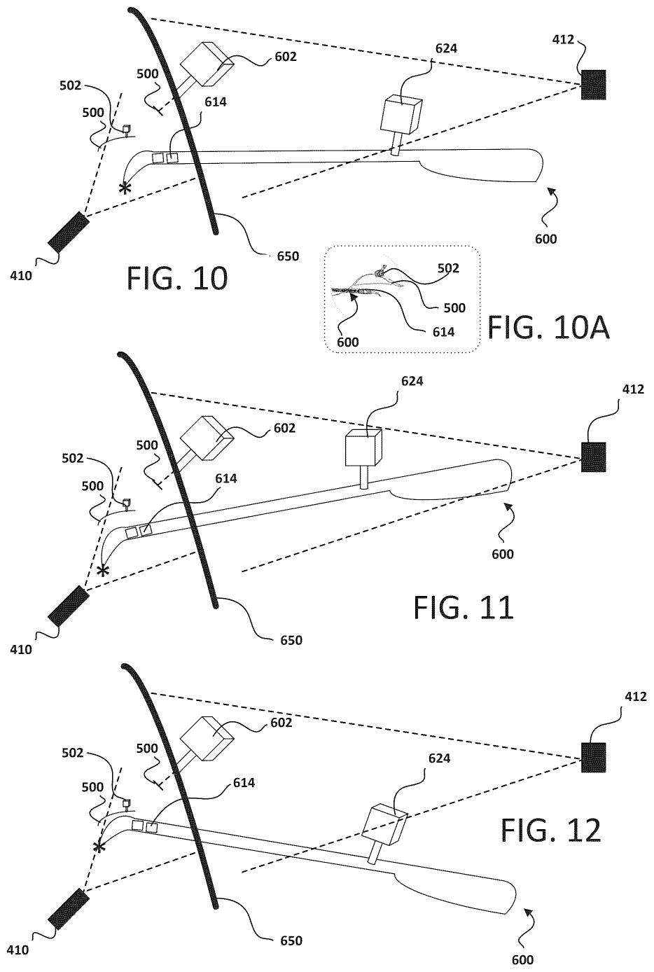

FIG. 10 is a slightly simplified version of FIG. 9 that shows an example position and orientation of tool 600 being held stationary while its tip in its distal portion and accordingly fiducial set 614 are at a first location within the surgical site. At this position and orientation, camera head 410 of the arthroscope (to the left of line 650 delineating patient anatomy) has within its field of view both fiducial set 614 and internal bone fiducial 502, itself affixed to bone 500. FIG. 10A shows an arthroscopic field of view containing tool 600, fiducial set 614, bone 500, and internal bone fiducial 502.

Also, at this position and orientation, external camera 412 (to the right of line 650 in FIG. 10) has within its field of view both fiducial set 624 and external bone fiducial 602, itself affixed to bone 500. For this position and orientation, at least a first image may be captured by camera 412 of the proximal portion 622, fiducial set 624, and external bone fiducial 602, simultaneously with the capture of at least a second image by camera head 410 of the distal portion 612, fiducial set 614, and internal bone fiducial 502. Capture of at least the first image and at least the second image captures the tip of tool 600 in the coordinate systems of fiducial sets 614 and 624 and with respect to respective bone fiducials 502 and 602. At least the first image may be processed to determine a first three-dimensional point of the tip in the external coordinate system, with the external bone fiducial 602 representing the origin of the external coordinate system. Also, at least the second image may be processed to determine a second three-dimensional point of the tip in the internal coordinate system, with the internal bone fiducial 502 representing the origin of the internal coordinate system. At the position and orientation of the tool 600 shown in FIG. 10, therefore, first and second three-dimensional points, each in respective ones of the external and internal coordinate systems, are captured for the first location.

FIG. 11 shows an example position and orientation of tool 600 being held stationary while its distal portion and fiducial set 614 are at a second location within the surgical site. At this position and orientation, camera head 410 of the arthroscope (to the left of line 650 delineating patient anatomy) has within its field of view both fiducial set 614 and internal bone fiducial 502, itself affixed to bone 500. Also, at this position and orientation, external camera 412 (to the right of line 650 in FIG. 11) has within its field of view both fiducial set 624 and external bone fiducial 602, itself affixed to bone 500. As in FIG. 10, at the position and orientation of the tool 600 shown in FIG. 11, therefore, first and second three-dimensional points of the tip of tool 600, each in respective ones of the external and internal coordinate systems, are captured for the second location.

FIG. 12 shows an example position and orientation of tool 600 being held stationary while its distal portion and fiducial set 614 are at a third location within the surgical site. At this position and orientation, camera head 410 of the arthroscope (to the left of line 650 delineating patient anatomy) has within its field of view both fiducial set 614 and internal bone fiducial 502, itself affixed to bone 500. Also, at this position and orientation, external camera 412 (to the right of line 650 in FIG. 12) has within its field of view both fiducial set 624 and external bone fiducial 602, itself affixed to bone 500. As in FIGS. 10 and 11, at the position and orientation of the tool 600 shown in FIG. 12, therefore, first and second three-dimensional points of the tip of tool 600, each in respective ones of the external and internal coordinate systems, are captured for the third location.

It will be appreciated that first, second, and third locations of the tip of tool 600 (or whatever specific point, if not the tip itself, such as another point on the tool or even off of the tool, whose location is being determined) may be random. While in this description just three locations are shown for brevity, it may be useful to capture pairs of first and second 3D points of the tip of tool 600 for a large number of additional locations so that there is sufficient data to generate a reliable mathematical transformation using the pairs of 3D points. In this example, the first 3D points in the internal coordinate system for all of the locations of the tip of tool 600 may be regarded as forming a first point cloud, and the second 3D points in the external coordinate system for all of the locations of the tip of tool 600 may be regarded as a second point cloud.

The above-described processes have been described without taking into account that the capturing of images by the arthroscopic camera may occur with a timing that is offset from the capturing of images by the external camera. More particularly, the above-described processes assume that either the capture of images by both cameras for the coordinate system merging is actually synchronized (using, for example, a hardware mechanism), or that there is a known offset between the timing (express, for example, in milliseconds and/or in frames) of receipt of the images, such that an image captured by the arthroscopic camera can be paired with an image captured by the external camera for determining respective 3D points, on the basis that the paired images are known to have been captured by the respective image sensors at the same time. One might consider that images in a pair of such images may not have been captured simultaneously, but that they were both captured while the tool 600 was held perfectly stationary in the same place for both such that they contain substantially the same data as those of the images actually captured simultaneously. However, since it can be practically very difficult to ensure that the tool 600 remains perfectly stationary, having information about an offset in capture timing and using that information to determine which of the internal images and the external images to pair with one another, is an important aspect.

An example scenario for internal/external camera systems is that the two systems may have different latencies due to respective different times for buffering an image, converting the image to the data transfer medium (e.g. USB cable or ethernet), sending the image over such a medium, reconstructing the image in a buffer, then finally presenting the image to a computer processor being tasked with processing it for fiducials. It may be the case that the external system's end-to-end latency is greater than that of an arthroscopic camera, due to it being a more external system with greater conversions, and even distance to traverse. It may alternatively be that the arthroscopic camera introduces more latency than the external camera. In either case, the amount of relative delay must be accounted for as an offset, in the form for example of a number of frames delayed from an actual event trigger or a number of milliseconds.

However, it will be appreciated that if tool 600 is being held in a given position while the arthroscopic camera captures a given first image or set of first images, and is thereafter moved by the time the external camera captures a given second image or set of second images, the actual position of fiducial 614 may have changed by the time the second image or set of second images is actually captured. It will be appreciated that, in embodiments, both the arthroscopic camera (or whichever first image capture system is used to capture internal images of the surgical site at the micro scale) and the external camera (or whichever second image capture system is used to capture external images of the surgical room at the macro scale) may capture images continuously as video frames. Each of the arthroscopic and external cameras may capture images at different frame rates and/or at different real time capture times. In order for a 3D point in the internal coordinate system to be paired with a 3D point in the external coordinate system to provide an accurate transformation, the surgical system should ensure that both 3D points were captured at the same time. As such, in embodiments an offset between timing of image capture by the external camera and timing of image capture by the arthroscopic camera may be determined, with the registration, or pairing, of 3D points captured by each of the cameras being done based on the offset between timings of capture. Furthermore, if the arthroscopic and external cameras have different frame rates (i.e., different rates of capture of respective frames) then the different rates must also be accounted for, again in order to ensure that frames to be paired are captured simultaneously. While it may be that tool 600 can be held sufficiently stationary such that contents in images in a pair captured at different times would be practically indistinguishable from those actually captured simultaneously, it may be challenging for a user or a system to reliably maintain tool 600 entirely stationary for a long enough time spans to practically achieve this. Thus, the system having knowledge of any offset in capture timing, latency, difference in frame rate etc. may reduce or eliminate the requirement that tool 600 be held stationary for very long.

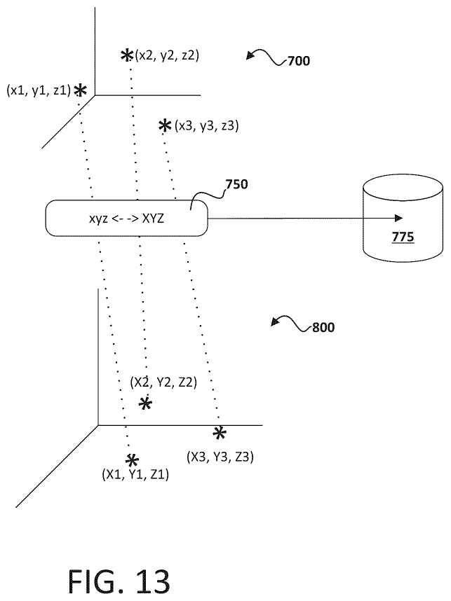

FIG. 13 shows a representation of an internal coordinate system 700 having three (3) captured 3D points (x1,y1,z1); (x2,y2,x2); and (x3,y3,z3), and an external coordinate system 800 having three respective captured 3D points (X1, Y1, Z1); (X2, Y2, Z2); and (X3, Y3, Z3). Dotted lines show the pairings between the 3D points in the internal coordinate system 700 and their respective 3D point in the external coordinate system 800, corresponding to pairs of images from which the 3D points were gleaned. The box 750 represents a mathematical transformation between the internal 3D points and the external 3D points and, once generated based on the internal and external 3D points, may be stored in a datastore 775 that is part of or accessible to the surgical system for use in transforming between the internal and external coordinate systems as desired for surgical guidance.

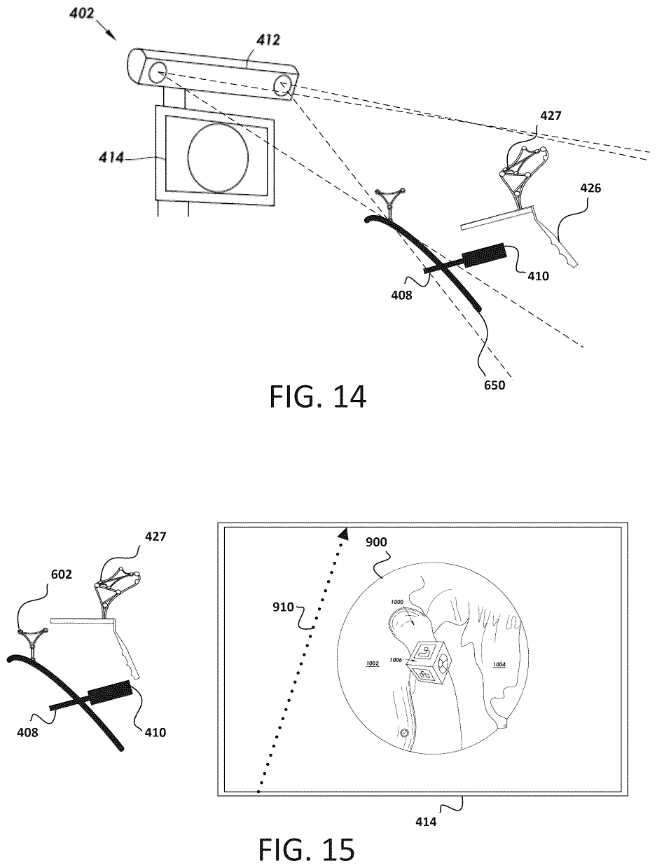

In an example, surgical guidance may be provided in relation to an arthroscopic, or internal, view based on tracking of objects in a surgical room external to the surgical site. Like FIG. 6, FIG. 14 shows a conceptual drawing of a surgical room with various objects within the surgical room, with the patient anatomy 650 delineating the surgical site internal to the patient from the surgical room external to the patient. In particular, visible to camera 412 in FIG. 6 is a proximate end of an arthroscope 408, a bone fiducial 602, an aimer 426, and an aimer fiducial 427.

FIG. 15 shows display 414 with an arthroscopic view 900 of a surgical site as in FIG. 14, as seen within the field of view of arthroscope 408. To the left of display 414 is a relational view of the position and orientation of drill fiducial 427 with respect to bone fiducial 502, as would be seen by external camera 412. This relational view is not that which would be displayed to a surgeon, as a surgeon would have aimer 426 in hand to manipulate in the surgical room. Rather, the relational view is shown in this figure to demonstrate how contents of display 414 may be presented depending on the position and orientation of aimer fiducial 427 with respect to bone fiducial 502. In particular, in this figure, a first indicia 910 in the form of a dotted line with an arrow is shown displayed on display 414, but not overlying arthroscopic view 900. Only a central circular portion of display 414 is taken up with arthroscopic view 900. First indicia 910 indicates the orientation of aimer 426 with respect to the arthroscopic view 900. The position and orientation of first indicia 910 is calculated in real-time or near real-time, or otherwise periodically, based on position and orientation of aimer fiducial 427 with respect to bone fiducial 602, and the mathematical transformation 750 calculated between the internal and external coordinate systems 700, 800. That is, even though no part of aimer 426 is visible in arthroscopic view 900 due to aimer 426 not being within the view of view of arthroscope 408, first indicia 910 may be calculated and displayed in conjunction with the arthroscopic view 900 being displayed on display 414 based on external camera 412 capturing aimer fiducial 427 and bone fiducial 602, the images/frames of the captured video being processed to determine the relative position and orientation of aimer fiducial 427 in the external coordinate system 800, and the relative position and orientation being transformed using the mathematical transform to the internal coordinate system 700. With the guidance of first indicia 910, a surgeon may be able to determine how to position and orient aimer 426 so that it coincides with the arthroscopic view.

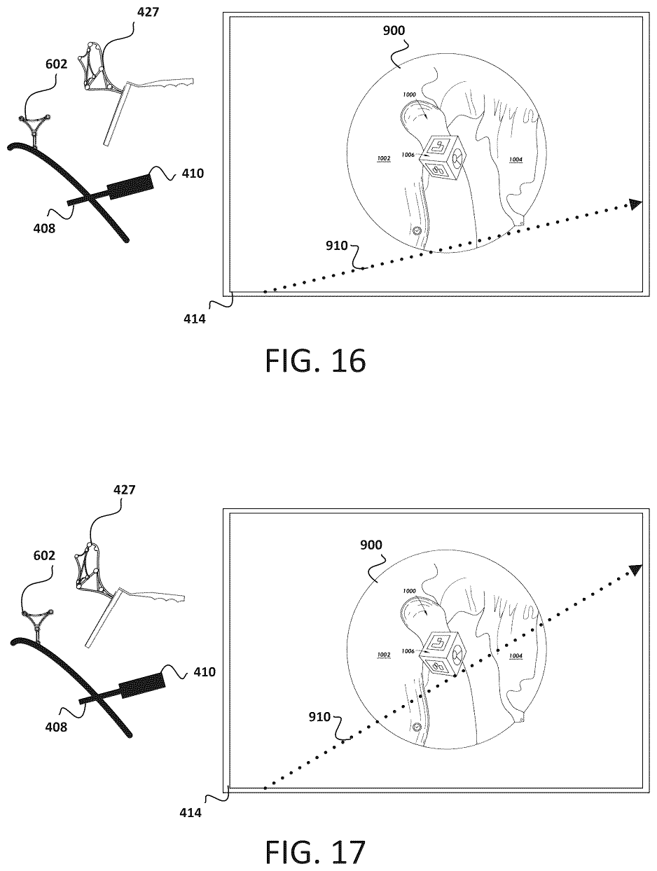

FIG. 16 shows display 414 with arthroscopic view 900 of a surgical site as in FIG. 15. To the left of display 414 is the relational view (again, not displayed to the surgeon but shown for ease of understanding) of the position and orientation of aimer fiducial 427 with respect to bone fiducial 502, as would be seen by external camera 412. In this figure, the position and/or orientation of aimer 426 has changed, which has caused a change in the position and/or orientation of aimer fiducial 427. As a result, and as described above, first indicia 910 is calculated to have a corresponding changed position and orientation of its own. It will be appreciated with reference to FIGS. 15 and 16, that a surgeon may move aimer 426 while viewing display 414 so as to observe how changes in position of aimer 426 affects first indicia 910. The surgeon may wish to cause first indicia 910 to at least coincide with arthroscopic view 900 so that the surgeon knows that advancing the aimer 426 along the trajectory may bring the drill wire 424 into the field of view of the arthroscopic camera 408 and thus into the arthroscopic view 900.

FIG. 17 shows display 414 with arthroscopic view 900 of a surgical site as in FIGS. 15 and 16. To the left of display 414 is the relational view (again, not displayed to the surgeon but shown for ease of understanding) of the position and orientation of drill fiducial 427 with respect to bone fiducial 502, as would be seen by external camera 412. In this figure, the position and/or orientation of aimer 426 has changed again, which has caused a change in the position and/or orientation of aimer fiducial 427. As a result, and as described above, first indicia 910 is calculated to have a corresponding changed position and orientation. In FIG. 17, first indicia 910 coincides with arthroscopic view 900 so that the surgeon knows that advancing the aimer 426 along the trajectory may bring the drill wire 424 into the field of view of the arthroscopic camera 408 and thus into the arthroscopic view 900.

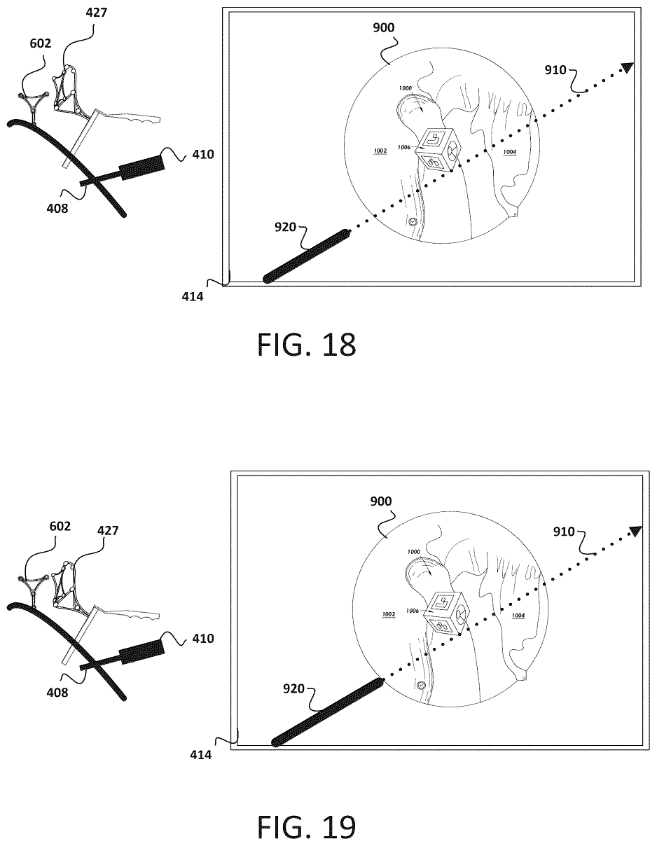

FIG. 18 shows display 414 with arthroscopic view 900 of a surgical site as in previous figures. To the left of display 414 is the relational view (again, not displayed to the surgeon but shown for ease of understanding) of the position and orientation of aimer fiducial 427 with respect to bone fiducial 502, as would be seen by external camera 412. In this figure, the position of aimer 426 has changed, but its orientation has remained the same as in FIG. 17. Aimer 426 has advanced further into the patient towards the surgical site. This has not caused a change in the position and/or orientation of first indicia 910. However, because aimer 426 has advanced to bring the drill wire 424 along the same trajectory but closer to the surgical site, a second indicia 920 is now displayed showing the relative position of the drill wire 424 to the arthroscopic view 900. In FIG. 17, first indicia 910 coincides with arthroscopic view 900 so that the surgeon knows that advancing the aimer 426 along the trajectory may bring the drill wire 424 into the field of view of the arthroscopic camera 408 and thus into the arthroscopic view 900, and second indicia 920 provides the surgeon with information about how much farther along the trajectory the drill wire 424 may been to be advanced to do so.

FIG. 19 shows display 414 with arthroscopic view 900 of a surgical site as in previous figures. To the left of display 414 is the relational view (again, not displayed to the surgeon but shown for ease of understanding) of the position and orientation of aimer fiducial 427 with respect to bone fiducial 502, as would be seen by external camera 412. In this figure, the position of aimer 426 has changed, but its orientation has remained the same as in FIG. 17. In particular, aimer 426 has advanced even further along the trajectory indicated by first indicia 910, to a position just outside of arthroscopic view 900 as indicated by second indicia 920.

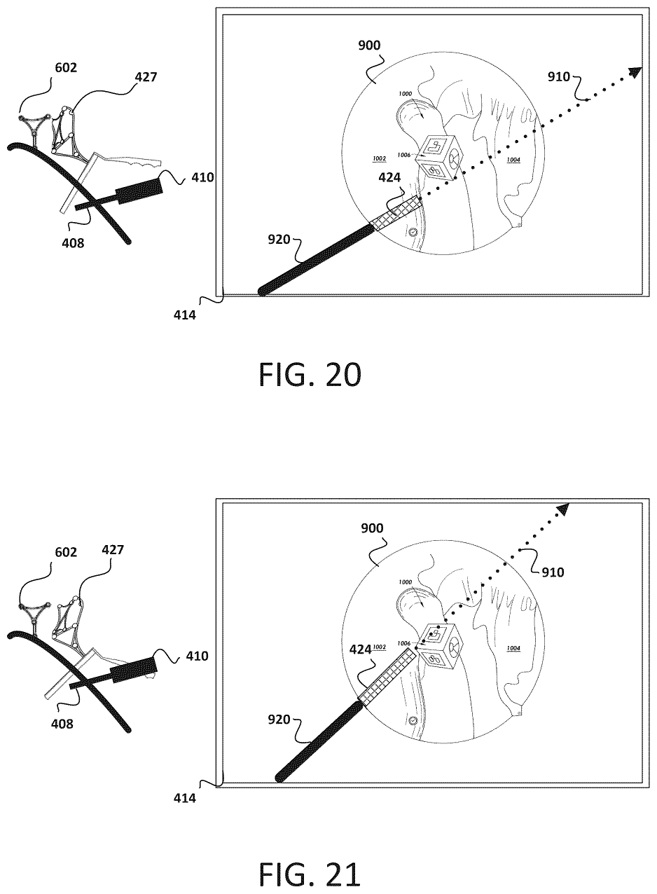

FIG. 20 shows display 414 with arthroscopic view 900 of a surgical site as in previous figures. To the left of display 414 is the relational view (again, not displayed to the surgeon but shown for ease of understanding) of the position and orientation of aimer fiducial 427 with respect to bone fiducial 502, as would be seen by external camera 412. In this figure, the position of aimer 426 has changed, but its orientation has remained the same as in FIG. 17. In particular, aimer 426 has advanced even further along the trajectory indicated by first indicia 910, to a position such that the end of drill wire 424 can be seen within the field of view of arthroscope 408, it having been brought to this position and orientation under the control of the surgeon with the surgical guidance provided as described above.

FIG. 21 shows display 414 with arthroscopic view 900 of a surgical site as in previous figures. To the left of display 414 is the relational view (again, not displayed to the surgeon but shown for ease of understanding) of the position and orientation of aimer fiducial 427 with respect to bone fiducial 502, as would be seen by external camera 412. In this figure, the position of aimer 426 has changed, as has its orientation. In particular, the position and orientation of first indicia 910 has changed according to the change in position and orientation of aimer 426, and aimer 426 has advanced even further along the new trajectory indicated by first indicia 910, to a position such that more of the end of drill wire 424 can be seen within the field of view of arthroscope 408.

Alternative approaches to registering internal and external coordinate systems are contemplated. For example instead of at least three (3) pairs of acquisitions from the image-capture systems generating respective point clouds of multiple 3D points that can, in turn, be processed so as to be registered to each other, an approach based on a single pair of acquisitions and a known Tₜₒₒₗ is possible. Such an approach assumes that the two image-capture systems (internal, external) are synchronized with each other or that there is at least a known timing offset between image captures of the two image-capture systems such that an image captured by a first of the image-capture systems can be paired with an image captured by the second of the image-capture systems on the basis that they were known to be captured simultaneously.

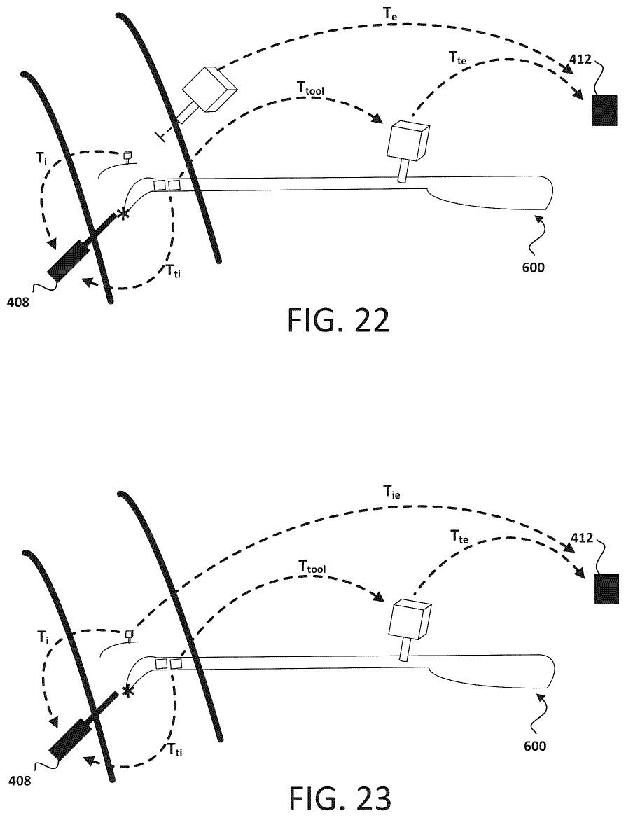

FIG. 22 shows an example of components of a surgical system arrangement for merging the internal and external coordinate systems with a set of transformations that may be combined to produce a spatial transformation between external and internal coordinate systems. In this example, a series of transformations are combined to determine a transformation between an internal bone fiducial which establishes the internal coordinate system, and an external bone fiducial which establishes the external coordinate system.

In this example, Tₜₒₒₗ is known and is available to the surgical controller 418, and is a transformation between the internal tool fiducial that is associated with the distal end of the tool 600 and the external tool fiducial that is associated with the proximal end of the tool 600, representing the known pose. A transformation Tₜᵢ represents a pose of the internal tool fiducial with respect to the arthroscope 408 (internal image-capture system), and a transformation Tᵢ represents a pose of the internal bone fiducial with respect to the arthroscope 408. Similarly, a transformation Tₜₑ represents a pose of the external tool fiducial with respect to the camera 412 (external image-capture system), and a transformation Tₑ represents a pose of the external bone fiducial with respect to the camera 412. With these multiple individual transformations having been defined, a complete transformation T from the internal tool fiducial to the external tool fiducial can be determined as in Equation (1), below:

$$T = {T}_{i} {T}_{t i}^{- 1} {T}_{tool} {T}_{t e} {T}_{e}^{- 1} \qquad ( 1 )$$

As explained herein, the above-explained approach is useful when external and internal image capture systems are synchronized, or at least when there is a known timing offset between their captures such that external and internal images can be paired for processing on the basis that they were known to be captured simultaneously. However, if the external and internal image capture systems are not synchronized, and there is not a known timing offset between their captures, then a method of determining a timing offset is useful. In this description, determining a timing offset may be conducted by tracking trajectories of 3D points traced using a tool such the tool 600, in a manner that processes series' of images (video frames) captured by each of the internal and external image-capture systems while tracing the tool through a trajectory. For example, for a short defined “calibration” time period a surgeon may be asked to trace the tool through a trajectory—such as a sinusoidal, square wave, or random walk shape—within the surgical site, while keeping both the internal tool fiducial and the internal bone fiducial within the field of view of the internal image-capture system as well as keeping the external tool fiducial and the external bone fiducial within the field of view of the external image-capture system. After this time period, the series' of images captured respectively by the internal and external image-capture systems of the respective trajectories of the internal and external tool fiducials with respect to their respective bone fiducials may be processed to identify counterpart trajectories in the two series so that two sets of points, with associated timestamps, can be reconstructed. Once the counterpart trajectories have been identified, their starting and ending points and/or other uniquely-identifiable points may each be reconstructed. For example, the endpoint of a trajectory in the series of images captured by the internal image-capture system may “appear” in a FRAME INT<x> (i.e. internal frame number x), whereas the endpoint of the corresponding trajectory in the series of images captured by the external image-capture system may “appear” in a FRAME EXT<i> (i.e., external frame number i). This information can be used to determine that FRAME INT<x> was captured at the same actual time as FRAME EXT<i>, and to extrapolate that determination based on frequency of image captures of each image-capture system and perhaps other known factors to derive the timing offset for, in turn, pairing images during coordinate system registration as described herein. Furthermore, by processing the two series of images to match at the level of abstraction of trajectories of internal and external tool fiducials through respective series, one may work backwards by matching points along the respective trajectories (such as matching the determined starting point of the trajectory in the series captured by the external image-capture system to the determined starting point of the trajectory in the series captured by the internal image-capture system) and use the actual points as the pairs of 3D points useful for actually generating a transformation from the internal coordinate system to the external coordinate system without necessarily embarking on a further discrete registration process. That is, by tracing a trajectory with the tool 600 and first matching trajectories occurring in the two image series, the processing structure can work backwards to both determine the timing offset and to generate point clouds between which a coordinate system transformation can be determined.

For use in a method of surgical navigation, it may be useful to obtain and use a transformation between internal and external coordinate systems without fixing an external bone fiducial 614 to a patient. FIG. 23 shows another example of components of a surgical system arrangement for merging internal and external coordinate systems with a set of transformations that may be combined to produce a spatial transformation between the external and internal coordinate systems. In this example, a series of transformations are combined to determine a transformation between an internal bone fiducial which establishes the internal coordinate system, and an external camera 412 which itself establishes the external coordinate system instead of a fixed external bone fiducial. In embodiments, the external camera 412 may be a head mounted display (HMD) that moves with the head of the surgeon as he or she works in the surgical room, faces the surgical site, and otherwise studies and conducts surgical operations. It may be useful for the surgeon, using the HMD, to have computer-assisted surgical navigation that conducts a frame-by-frame (or, more generally, real-time, near-real-time, or otherwise usefully periodic) transformation calculation from the internal coordinate system to the HMD itself. This may enable the surgeon to be provided with overlays, indicia, and other guidance thereby to see into the surgical site as though peering from the outside through the skin into the surgical site of the patient. Therefore, rather than a single transformation between an internal bone fiducial and an external bone fiducial for the whole of the surgical procedure, the surgical system establishes a set of transformations that must be updated regularly—such as for every frame or every few frames-in order to ensure that the current orientation of the HMD with respect to the surgical site is reflected in the transformation.

In this example, as in others, Tₜₒₒₗ is known and is a transformation between the internal tool fiducial that is associated with the distal end of the tool 600 and the external tool fiducial that is associated with the proximal end of the tool 600, representing the known pose. For each of a pair of first video frames captured simultaneously by the first, or external, image-capture system and by the second, or internal, image-capture system, a transformation Tₜᵢ calculated by processing the second video frame of the pair and represents a current first internal pose of the internal tool fiducial (associated with the distal portion of the tool) in the internal coordinate system i.e., with respect to the arthroscope 408. A transformation Tᵢ represents a current second internal pose of the internal bone fiducial in the internal coordinate system i.e., with respect to the arthroscope 408. A third internal pose of an internal object also captured within the second video frames is also determined with respect to the internal coordinate system. Similarly, a transformation Tₜₑ represents a current external pose of the external tool fiducial with respect to the camera 412 (external image-capture system), and a transformation Tᵢₑ represents a pose of the internal bone fiducial with respect to the camera 412 (the external coordinate system) that must be updated regularly through the surgical procedure. With these multiple individual transformations having been defined, the transformation Tᵢₑ from the internal bone fiducial to the camera 412 (such as an HMD) can be determined as in Equation (2), below:

$${T}_{i e} = {T}_{i} {T}_{t i}^{- 1} {T}_{tool} {T}_{t e} \qquad ( 2 )$$

With Tᵢₑ being determined regularly (such as frame-by-frame, or every few frames) during a surgical procedure, the position of a tool or other internal object with respect to the internal bone fiducial having the internal coordinate system may be transformed to a position with respect to the external camera 412 having the external coordinate system. With this transformation, an indicia corresponding to the tool with respect to the internal bone fiducial can be displayed in the frame of reference of the external camera 412. By not requiring an external bone fiducial for this, there is the potential for a decrease in the risk of complications and recovery time for the patient.

Various applications of registering internal and external coordinate systems may be implemented.

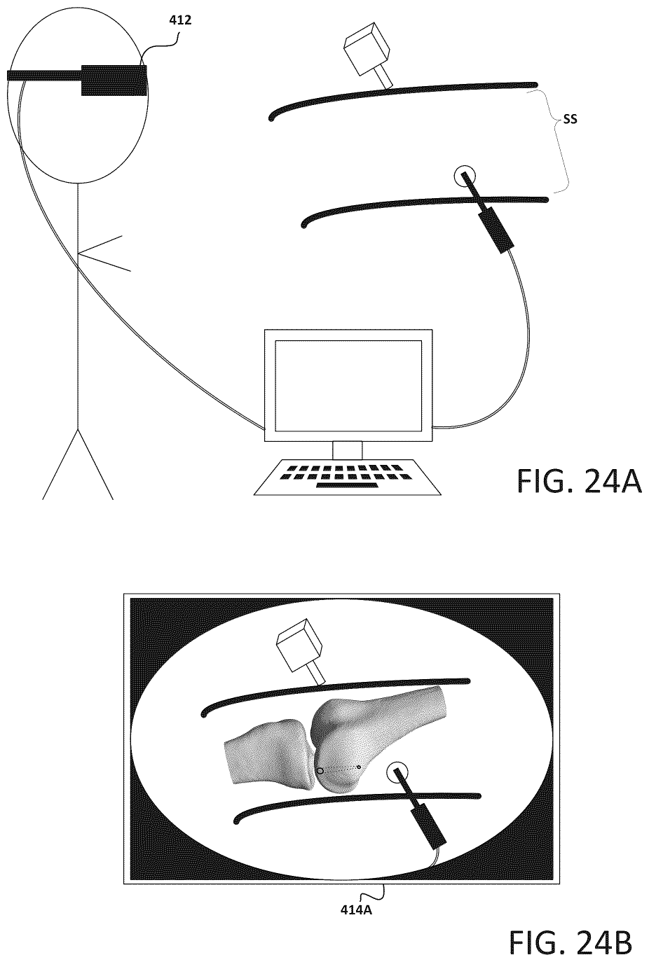

For example, FIG. 24A shows components of a surgical system in which a user is wearing a head mounted display (HMD) having a camera 412 capturing an external bone fiducial and portions of the exterior of the patient anatomy. With an arthroscope 408 having a field of view within the surgical site, as shown in FIG. 24A, the user's own display 414A may display what is captured directly by camera 412 in its field of view of the exterior of the surgical site, but may also display an overlay showing a 3D bone model registered to the bone, and a planned bone tunnel positioned with respect to the 3D bone model, as shown in FIG. 24B. In this way, the user is provided with the impression he or she is seeing through the skin of the patient, and is provided with useful positional and relational visual information about the relative location of the bone and the planned tunnel.

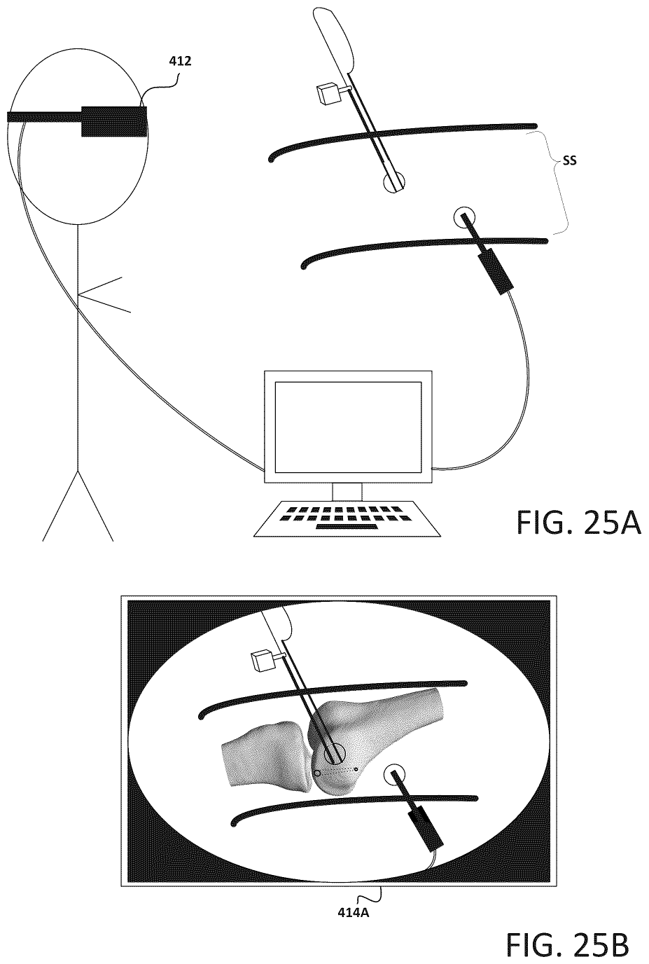

As another example, FIG. 25A shows components of a surgical system in which a user is wearing a head mounted display (HMD) having a camera 412 capturing an external tool fiducial and portions of the exterior of the patient anatomy, with no external bone fiducial. With an arthroscope 408 having a field of view within the surgical site, as shown in FIG. 25A, the user's own display 414A may display what is captured directly by camera 412 in its field of view of the exterior of the surgical site, but may also display an overlay showing a 3D bone model registered to the bone, and a planned bone tunnel positioned with respect to the 3D bone model, as shown in FIG. 25B. In this way, the user is provided with the impression he or she is seeing through the skin of the patient, and is provided with useful positional and relational visual information about the relative location of the bone and the planned tunnel.

The kinds of visualizations enabled by a registration between internal and external coordinate systems may have many uses and benefits, for example in enabling a surgeon to see the quality of registration of a bone model to the bone, for example seeing if a femoral head is correctly aligned so as to provide the surgeon with assurance as to the registration, or informing the surgeon as to a need to modify the bone mode registration itself.

Other applications may include surgical procedures in which instruments are tracked externally, such as in guided osteotomy, in which cutting the bone and placing screws and/or needles is performed using outside visualization rather than purely arthroscopically. Applications for registration of internal and external coordinate systems may involve robotic surgery, in which for example positions of the robotically controlled tool during a resection of bone may be accurately tracked by using feedback obtained by tracking an external fiducial of the robotically controlled tool.

### Software and Hardware

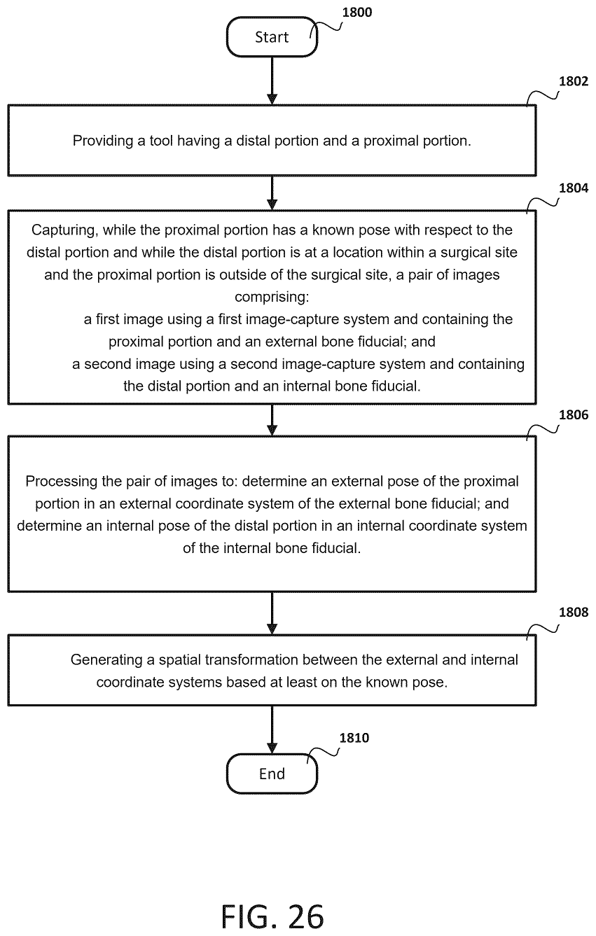

FIG. 26 shows a method for registering internal and external coordinate systems of a surgical system, in accordance with at least some embodiments. In particular, the method starts (block 1800) and comprises: providing a tool having a distal portion and a proximal portion (block 1802); capturing, while the proximal portion has a known pose with respect to the distal portion and while the distal portion is at a location within a surgical site and the proximal portion is outside of the surgical site, a pair of images comprising: a first image using a first image-capture system and containing the proximal portion and an external bone fiducial; and a second image using a second image-capture system and containing the distal portion and an internal bone fiducial (block 1804); processing the pair of images to: determine an external pose of the proximal portion in an external coordinate system of the external bone fiducial; and determine an internal pose of the distal portion in an internal coordinate system of the internal bone fiducial (block 1806); and generating a spatial transformation between the external and internal coordinate systems based at least on the known pose (block 1808). Thereafter, the method ends (block 1810). The example method may be implemented by computer instructions executed with the processor of computer system, such as the surgical controller 418 (FIG. 4).

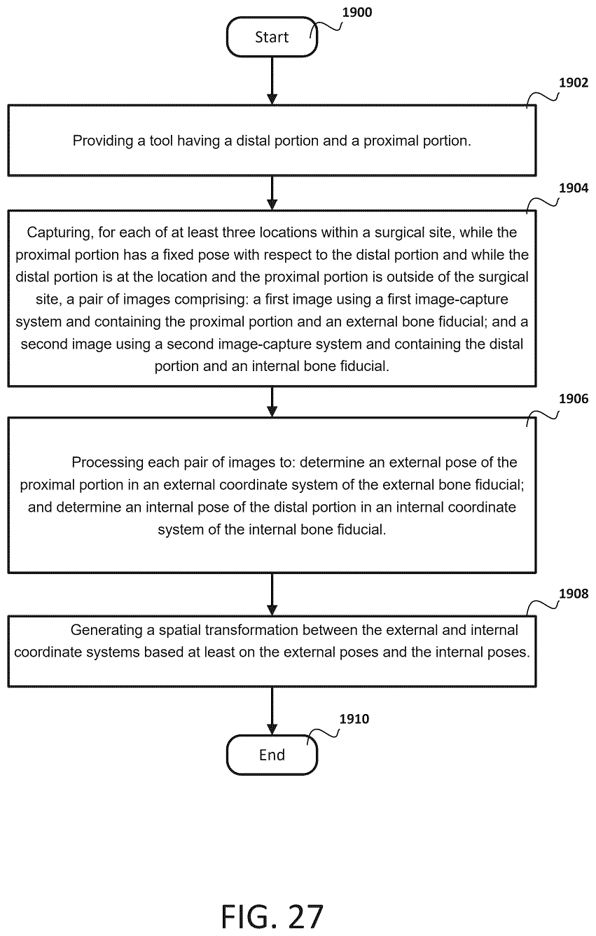

FIG. 27 shows a method for registering internal and external coordinate systems of a surgical system, in accordance with at least some embodiments. In particular, the method starts (block 1900) and comprises: providing a tool having a distal portion and a proximal portion (block 1902); capturing, for each of at least three locations within a surgical site, while the proximal portion has a fixed pose with respect to the distal portion and while the distal portion is at the location and the proximal portion is outside of the surgical site, a pair of images comprising: a first image using a first image-capture system and containing the proximal portion and an external bone fiducial; and a second image using a second image-capture system and containing the distal portion and an internal bone fiducial (block 1904); processing each pair of images to: determine an external pose of the proximal portion in an external coordinate system of the external bone fiducial; and determine an internal pose of the distal portion in an internal coordinate system of the internal bone fiducial (block 1906); and generating a spatial transformation between the external and internal coordinate systems based at least on the external poses and the internal poses (block 1908). Thereafter, the method ends (block 1910). The example method may be implemented by computer instructions executed with the processor of computer system, such as the surgical controller 418 (FIG. 4).

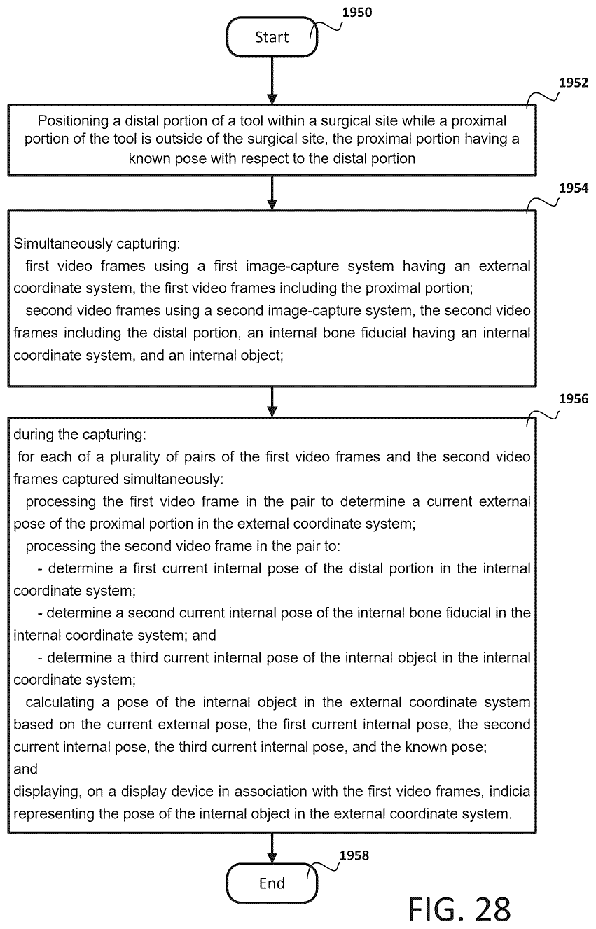

FIG. 28 shows a method for surgical navigation, in accordance with at least some embodiments. In particular, the method starts (block 1950) and comprises: positioning a distal portion of a tool within a surgical site while a proximal portion of the tool is outside of the surgical site, the proximal portion having a known pose with respect to the distal portion (step 1952); simultaneously capturing: first video frames using a first image-capture system having an external coordinate system, the first video frames including the proximal portion; and second video frames using a second image-capture system, the second video frames including the distal portion, an internal bone fiducial having an internal coordinate system, and an internal object (step 1954); and during the capturing: for each of a plurality of pairs of the first video frames and the second video frames captured simultaneously: processing the first video frame in the pair to determine a current external pose of the proximal portion in the external coordinate system; processing the second video frame in the pair to: determine a first current internal pose of the distal portion in the internal coordinate system; determine a second current internal pose of the internal bone fiducial in the internal coordinate system; and determine a third current internal pose of the internal object in the internal coordinate system; calculating a pose of the internal object in the external coordinate system based on the current external pose, the first current internal pose, the second current internal pose, the third current internal pose, and the known pose; and displaying, on a display device in association with the first video frames, indicia representing the pose of the internal object in the external coordinate system (step 1956).

Thereafter, the method ends (block 1958). The example method may be implemented by computer instructions executed with the processor of computer system, such as the surgical controller 418 (FIG. 4).

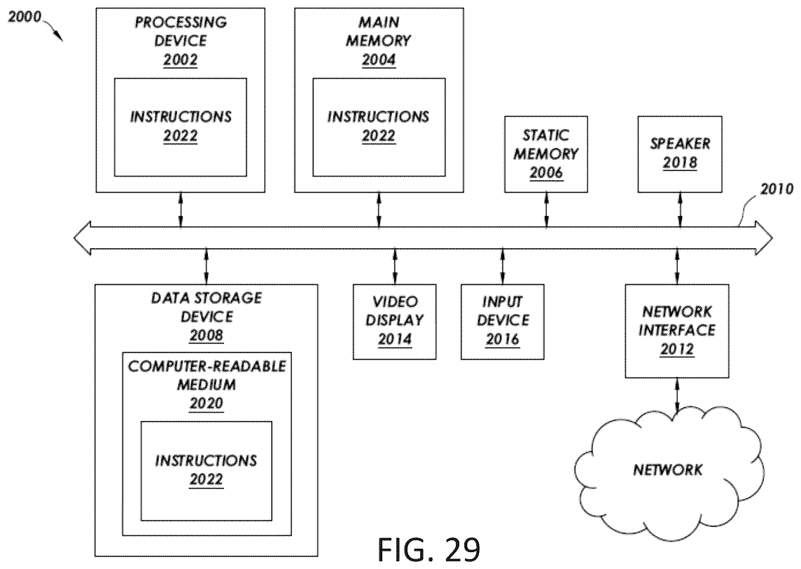

FIG. 29 shows an example computer system 2000. In one example, computer system 2000 may correspond to the surgical controller 418, a tablet device within the surgical room, or any other system that implements any or all the various methods discussed in this specification as part of a surgical system. The computer system 2000 may be connected (e.g., networked) to other computer systems in a local-area network (LAN), an intranet, and/or an extranet (e.g., device cart 402 network), or at certain times the Internet (e.g., when not in use in a surgical procedure). The computer system 2000 may be a server, a personal computer (PC), a tablet computer or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 2000 includes a processing device 2002, a main memory 2004 (e.g., read-only memory (ROM), flash memory, dynamic random access memory (DRAM) such as synchronous DRAM (SDRAM)), a static memory 2006 (e.g., flash memory, static random access memory (SRAM)), and a data storage device 2008, which communicate with each other via a bus 2010.

Processing device 2002 represents one or more general-purpose processing devices such as a microprocessor, central processing unit, or the like. More particularly, the processing device 2002 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 2002 may also be one or more special-purpose processing devices such as an application specific integrated circuit (ASIC), a field programmable gate array (FPGA), a digital signal processor (DSP), network processor, or the like. The processing device 2002 is configured to execute instructions for performing any of the operations and steps discussed herein. Once programmed with specific instructions, the processing device 2002, and thus the entire computer system 2000, becomes a special-purpose device, such as the surgical controller 418.

The computer system 2000 may further include a network interface device 2012 for communicating with any suitable network (e.g., the device cart 402 network). The computer system 2000 also may include a video display 2014 (e.g., display device 414), one or more input devices 2016 (e.g., a microphone, a keyboard, and/or a mouse), and one or more speakers 2018. In one illustrative example, the video display 2014 and the input device(s) 2016 may be combined into a single component or device (e.g., an LCD touch screen).

The data storage device 2008 may include a computer-readable storage medium 2020 on which the instructions 2022 (e.g., implementing any methods and any functions performed by any device and/or component depicted described herein) embodying any one or more of the methodologies or functions described herein is stored. The instructions 2022 may also reside, completely or at least partially, within the main memory 2004 and/or within the processing device 2002 during execution thereof by the computer system 2000. As such, the main memory 2004 and the processing device 2002 also constitute computer-readable media. In certain cases, the instructions 2022 may further be transmitted or received over a network via the network interface device 2012.

While the computer-readable storage medium 2020 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

While various embodiments have been described, alternatives are possible.

For example, while methods and systems have been described that make use of a single external image-capture device, multiple external image-capture devices may be used in a given surgical system, with various transformations between the surgical site and each and between each other enabling exchanges of information between multiple systems.

Furthermore, an external image-capture system may be a camera such as camera 412, but may alternatively or in some combination be a HMD, a see-through display, a robot vision device, a smart power-tool having imaging capabilities, an opto-tracker, a tablet, smartphone, or any other camera-type imaging device.

It will be appreciated that, even though the external image-capture system described herein captures optical images on which image processing is conducted, external systems that can use other technologies to detect the pose of an external bone fiducial and/or the pose of an external tool fiducial may be used in conjunction with an internal image-capture system to generate transformations. For example, a non-imaging optical or electromagnetic tracking system may be used on conjunction with fiducials of these different modes in order to determine poses of the fiducials with respect to the non-imaging or electromagnetic tracking systems.

While a particular tool 600 has been described, alternatives are possible. For example, an alternative tool 600 may be without a tip extending beyond an internal tool fiducial. Alternatively or in some combination, a given tool may enable a user to change the relative pose between the internal tool fiducial and the external tool fiducial for various uses. However, provided that a fixed pose (whether known or not known) between the internal tool fiducial of the tool and the external tool fiducial of the tool is available for use and is fixed during the registration of the internal and external coordinate systems, and as long as the fiducials can be detected by respective tracking/imaging systems, various configurations of tools suitable for insertion into a surgical site may be used.

Furthermore, while internal tool fiducials and external tool fiducials have been described herein as particularly designed for recognition by respective tracking or image-capture systems, alternatives are possible. For example, a given image-capture system or the surgical system may be programmed to, or have a machine learning system trained to, reliably recognize features of a distal portion of the tool in images captured by the internal image-capture system in such a manner as to be able to discern, and determine the pose of, the distal portion with respect to an internal bone fiducial. Similarly, a proximal end of a tool may be discerned in images captured by an external image-capture system such that its pose with respect to the external bone fiducial may be reliably determined.

A given fiducial may have a different shape, such as instead of a fiducial being cubic in shape as in examples herein, the fiducial may have fewer or more sides than a cube and/or have some sides longer than others.

Also, where there may be multiple external sensors, whether they all be one kind of external sensor (for example, all visual, all optical or all electromagnetic), or whether they be different kinds of external sensors (for example, one visual and one optical, or one visual and one electromagnetic, or one optical and one electromagnetic, one visual, or one optical and one electromagnetic, or multiples of one and singles of the other, or any other combinations of multiple external sensors), the methods described herein may be applied to the multiple external sensors such that the internal and the one or more external sensor systems may be synchronized and/or aligned, allowing the exchange of information between the internal system and more than just one external system.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

### CLAUSES

Clause 1. A method for registering internal and external coordinate systems of a surgical system, the method comprising:
   providing a tool having a distal portion and a proximal portion;
   capturing, while the proximal portion has a known pose with respect to the distal portion and while the distal portion is at a location within a surgical site and the proximal portion is outside of the surgical site, a pair of images comprising:
a first image using a first image-capture system and containing the proximal portion and an external bone fiducial; and
a second image using a second image-capture system and containing the distal portion and an internal bone fiducial;
processing the pair of images to:
determine an external pose of the proximal portion in an external coordinate system of the external bone fiducial; and
determine an internal pose of the distal portion in an internal coordinate system of the internal bone fiducial;
and
generating a spatial transformation between the external and internal coordinate systems based at least on the known pose.
Clause 2. The method of clause 1, further comprising:
capturing internal video frames of at least one object and the internal bone fiducial using the second image-capture system;
based on the spatial transformation, processing the internal video frames to determine a pose of the at least one object in the external coordinate system; and
displaying, on a display device in association with external video frames captured using the first image-capture system, indicia representing the pose of the at least one object in the external coordinate system.
Clause 3. The method of clause 1, further comprising:
capturing external video frames of at least one object and the external bone fiducial using the first image-capture system;
based on the spatial transformation, processing the external video frames to determine a pose of the at least one object in the internal coordinate system; and
displaying, on a display device in association with internal video frames captured using the second image-capture system, indicia representing the pose of the at least one object in the internal coordinate system.
Clause 4. The method of clause 1, further comprising:
forming the pair of images by simultaneously capturing the first image and the second image.
Clause 5. The method of clause 4, wherein the forming is conducted based at least on a known offset between a time of image capture by the first image-capture system and a time of image capture by the second image-capture system.
Clause 6. The method of clause 4, wherein the forming is conducted based at least on a known rate of image capture by the first image-capture system and a known rate of image-capture by the second image-capture system.
Clause 7. The method of clause 1, wherein the second image-capture system comprises at least one of: an endoscopic camera and a needle scope.
Clause 8. The method of clause 1, wherein the first image-capture system comprises at least one of: a visual camera and an infrared camera.
Clause 9. The method of clause 1, wherein the first image includes an external tool fiducial associated with the proximal portion, and wherein processing the pair of images to determine the external pose of the proximal portion in the external coordinate system of the external bone fiducial comprises:
processing the first image to determine the external pose of the external tool fiducial in the external coordinate system of the external bone fiducial.
Clause 10. The method of clause 1, wherein the second image includes an internal tool fiducial associated with the distal portion, and wherein processing the pair of images to determine the internal pose of the distal portion in the internal coordinate system of the internal bone fiducial comprises:
processing the second image to determine the internal pose of the internal tool fiducial in the internal coordinate system of the internal bone fiducial.
Clause 11. A surgical system comprising:
a tool comprising a distal portion and a proximal portion, the proximal portion having a known pose with respect to the distal portion, the distal portion dimensioned to be received within a surgical site while the proximal portion is outside of the surgical site;
a first image-capture system outside of the surgical site;
   a second image-capture system inside the surgical site;
   processing structure comprising at least one computer processor, the processing structure in communication with the first image-capture system and the second image-capture system and configured for:
capturing, while the distal portion is at a location within the surgical site and the proximal portion is outside of the surgical site, a pair of images comprising:
a first image using the first image-capture system and containing the proximal portion and an external bone fiducial; and
a second image using the second image-capture system and containing the distal portion and an internal bone fiducial;
processing the pair of images to:
determine an external pose of the proximal portion in an external coordinate system of the external bone fiducial; and
determine an internal pose of the distal portion in an internal coordinate system of the internal bone fiducial;
and
generating a spatial transformation between the external and internal coordinate systems based at least on the known pose.
Clause 12. The surgical system of clause 11, the processing structure configured for:
capturing internal video frames of at least one object and the internal bone fiducial using the second image-capture system;
based on the spatial transformation, processing the internal video frames to determine a pose of the at least one object in the external coordinate system; and
displaying, on a display device in association with external video frames captured using the first image-capture system, indicia representing the pose of the at least one object in the external coordinate system.
Clause 13. The surgical system of clause 11, the processing structure configured for:
capturing external video frames of at least one object and the external bone fiducial using the first image-capture system;
based on the spatial transformation, processing the external video frames to determine a pose of the at least one object in the internal coordinate system; and
displaying, on a display device in association with internal video frames captured using the second image-capture system, indicia representing the pose of the at least one object in the internal coordinate system.
Clause 14. The surgical system of clause 11, the processing structure configured for:
forming the pair of images by simultaneously capturing the first image and the second image.
Clause 15. The surgical system of clause 14, wherein the processing structure is configured for conducting the forming based at least on a known offset between a time of image capture by the first image-capture system and a time of image capture by the second image-capture system.
Clause 16. The surgical system of clause 14, wherein the processing structure is configured for conducting the forming based at least on a known rate of image capture by the first image-capture system and a known rate of image-capture by the second image-capture system.
Clause 17. The surgical system of clause 11, wherein the second image-capture system comprises at least one of: an endoscopic camera and a needle scope.
Clause 18. The surgical system of clause 11, wherein the first image-capture system comprises at least one of: a visual camera and an infrared camera.
Clause 19. The surgical system of clause 11, wherein the first image includes an external tool fiducial associated with the proximal portion, and wherein processing the pair of images to determine the external pose of the proximal portion in the external coordinate system of the external bone fiducial comprises:
processing the first image to determine the external pose of the external tool fiducial in the external coordinate system of the external bone fiducial.
Clause 20. The surgical system of clause 11, wherein the second image includes an internal tool fiducial associated with the distal portion, and wherein processing the pair of images to determine the internal pose of the distal portion in the internal coordinate system of the internal bone fiducial comprises:
processing the second image to determine the internal pose of the internal tool fiducial in the internal coordinate system of the internal bone fiducial.
Clause 21. A method for registering internal and external coordinate systems of a surgical system, the method comprising:
   providing a tool having a distal portion and a proximal portion;
   capturing, for each of at least three locations within a surgical site, while the proximal portion has a fixed pose with respect to the distal portion and while the distal portion is at the location and the proximal portion is outside of the surgical site, a pair of images comprising:
a first image using a first image-capture system and containing the proximal portion and an external bone fiducial; and
a second image using a second image-capture system and containing the distal portion and an internal bone fiducial;
processing each pair of images to:
determine an external pose of the proximal portion in an external coordinate system of the external bone fiducial; and
determine an internal pose of the distal portion in an internal coordinate system of the internal bone fiducial;
and
generating a spatial transformation between the external and internal coordinate systems based at least on the external poses and the internal poses.
Clause 22. The method of clause 21, further comprising:
capturing video frames of at least one object and the internal bone fiducial using the second image-capture system;
based on the spatial transformation, processing the video frames to determine a pose of the at least one object in the external coordinate system; and
displaying, on a display device in association with video frames captured using the first image-capture system, indicia representing the pose of the at least one object in the external coordinate system.
Clause 23. The method of clause 21, further comprising:
capturing video frames of at least one object and the external bone fiducial using the first image-capture system;
based on the spatial transformation, processing the video frames to determine a pose of the at least one object in the internal coordinate system; and
displaying, on a display device in association with video frames captured using the second image-capture system, indicia representing the pose of the at least one object in the internal coordinate system.
Clause 24. The method of clause 21, further comprising:
forming each pair by pairing those of the at least one first and second images that were captured simultaneously.
Clause 25. The method of clause 24, wherein the forming is conducted based on a known offset between a time of image capture by the first image-capture system and a time of image capture by the second image-capture system.
Clause 26. The method of clause 24, wherein the forming is conducted based at least on a known rate of image capture by the first image-capture system and a known rate of image capture by the second image-capture system.
Clause 27. The method of clause 21, wherein generating the spatial transformation between the external and internal coordinate systems comprises:
   registering each of the locations in the external coordinate system to a 3D bone model thereby to generate a first bone model transformation;
   registering each of the locations in the internal coordinate system to the 3D bone model thereby to generate a second bone model transformation; and
   generating the spatial transformation between the external and internal coordinate systems based at least on the first bone model transformation and the second bone model transformation.
Clause 28. The method of clause 21, wherein the second image-capture system comprises at least one of: an endoscopic camera and a needle scope.
Clause 29. The method of clause 21, wherein the first image-capture system comprises at least one of: a visual camera and an infrared camera.
Clause 30. The method of clause 21, wherein each first image includes an external tool fiducial associated with the proximal portion, and wherein processing each pair of images to determine each external pose of the proximal portion in the external coordinate system of the external bone fiducial comprises:
processing each first image to determine each external pose of the external tool fiducial in the external coordinate system of the external bone fiducial.
Clause 31. The method of clause 21, wherein each second image includes an internal tool fiducial associated with the distal portion, and wherein processing each pair of images to determine each internal pose of the distal portion in the internal coordinate system of the internal bone fiducial comprises:
processing each second image to determine each internal pose of the internal tool fiducial in the internal coordinate system of the internal bone fiducial.
Clause 32. A surgical system comprising:
a tool comprising a distal portion and a proximal portion, the proximal portion having a fixed pose with respect to the distal portion, the distal portion dimensioned to be received within a surgical site while the proximal portion is outside of the surgical site;
a first image-capture system outside of the surgical site;
   a second image-capture system inside the surgical site; and
   processing structure comprising at least one computer processor, the processing structure in communication with the first image-capture system and the second image-capture system and configured for:
capturing, for each of at least three locations within the surgical site, while the distal portion of the tool is at the location and the proximal portion of the tool is outside of the surgical site, a pair of images comprising:
a first image using the first image-capture system and containing the proximal portion and an external bone fiducial; and
a second image using the second image-capture system and containing the distal portion and an internal bone fiducial;
and
processing each pair of images to:
determine an external pose of the proximal portion in an external coordinate system of the external bone fiducial; and
determine an internal pose of the distal portion in an internal coordinate system of the internal bone fiducial;
the processing structure further configured for:
generating a spatial transformation between the external and internal coordinate systems based at least on the external poses and the internal poses.
Clause 33. The surgical system of clause 32, the processing structure configured for:
capturing video frames of at least one object and the internal bone fiducial using the second image-capture system;
based on the spatial transformation, processing the video frames to determine a pose of the at least one object in the external coordinate system; and
displaying, on a display device in association with video frames captured using the first image-capture system, indicia representing the pose of the at least one object in the external coordinate system.
Clause 34. The surgical system of clause 32, the processing structure configured for:
capturing video frames of at least one object and the external bone fiducial using the first image-capture system;
based on the spatial transformation, processing the video frames to determine a pose of the at least one object in the internal coordinate system; and
displaying, on a display device in association with video frames captured using the second image-capture system, indicia representing the pose of the at least one object in the internal coordinate system.
Clause 35. The surgical system of clause 32, the processing structure configured for:
forming each pair by pairing those of the at least one first and second images that were captured simultaneously.
Clause 36. The surgical system of clause 35, wherein the forming is conducted based on a known offset between a time of image capture by the first image-capture system and a time of image capture by the second image-capture system.
Clause 37. The surgical system of clause 35, wherein the forming is conducted based at least on a known rate of image capture by the first image-capture system and a known rate of image capture by the second image-capture system.
Clause 38. The surgical system of clause 32, wherein for generating the spatial transformation between the external and internal coordinate systems the processing structure is configured for:
   registering each of the locations in the external coordinate system to a 3D bone model thereby to generate a first bone model transformation;
   registering each of the locations in the internal coordinate system to the 3D bone model thereby to generate a second bone model transformation; and
   generating the spatial transformation between the external and internal coordinate systems based at least on the first bone model transformation and the second bone model transformation.
Clause 39. The surgical system of clause 32, wherein the second image-capture system comprises at least one of: an endoscopic camera and a needle scope.
Clause 40. The surgical system of clause 32, wherein the first image-capture system comprises at least one of: a visual camera and an infrared camera.
Clause 41. The surgical system of clause 32, wherein each first image includes an external tool fiducial associated with the proximal portion, and wherein for processing each pair of images to determine the external pose of the proximal portion in the external coordinate system of the external bone fiducial the processing structure is configured for:
processing each first image to determine the external pose of the external tool fiducial in the external coordinate system of the external bone fiducial.
Clause 42. The surgical system of clause 32, wherein each second image includes an internal tool fiducial associated with the distal portion, and wherein for processing each pair of images to determine the internal pose of the distal portion in the internal coordinate system of the internal bone fiducial the processing structure is configured for:
processing each second image to determine the internal pose of the internal tool fiducial in the internal coordinate system of the internal bone fiducial.
Clause 43. A method of surgical navigation comprising:
   positioning a distal portion of a tool within a surgical site while a proximal portion of the tool is outside of the surgical site, the proximal portion having a known pose with respect to the distal portion;
   simultaneously capturing:
first video frames using a first image-capture system having an external coordinate system, the first video frames including the proximal portion; and
second video frames using a second image-capture system, the second video frames including the distal portion, an internal bone fiducial having an internal coordinate system, and an internal object;
   and
during the capturing:
for each of a plurality of pairs of the first video frames and the second video frames captured simultaneously:
processing the first video frame in the pair to determine a current external pose of the proximal portion in the external coordinate system;
processing the second video frame in the pair to:
determine a first current internal pose of the distal portion in the internal coordinate system;
determine a second current internal pose of the internal bone fiducial in the internal coordinate system; and
determine a third current internal pose of the internal object in the internal coordinate system;
calculating a pose of the internal object in the external coordinate system based on the current external pose, the first current internal pose, the second current internal pose, the third current internal pose, and the known pose;
and
displaying, on a display device in association with the first video frames, indicia representing the pose of the internal object in the external coordinate system.
Clause 44. The method of clause 43, wherein the second image-capture system comprises at least one of: an endoscopic camera and a needle scope.
Clause 45. The method of clause 43, wherein the first image-capture system comprises at least one of: a visual camera and an infrared camera.
Clause 46. The method of clause 45, where the visual camera and the display device are components of a head-mounted-display (HMD) device.
Clause 47. The method of clause 43, wherein processing the first video frame to determine a current external pose of the proximal portion in the external coordinate system comprises:
processing the first video frame to determine the external pose of an external tool fiducial that is associated with the proximal portion.
Clause 48. The method of clause 43, wherein processing the second video frame to determine a current internal pose of the distal portion in the internal coordinate system comprises:
processing the second video frame to determine the internal pose of an internal tool fiducial that is associated with the distal portion.
Clause 49. A surgical system comprising:
a tool comprising a distal portion and a proximal portion, the proximal portion having a known pose with respect to the distal portion, the distal portion dimensioned to be received within a surgical site while the proximal portion is outside of the surgical site;
a first image-capture system outside of the surgical site and having an external coordinate system;
   a second image-capture system inside the surgical site; and
   processing structure comprising at least one computer processor, the processing structure in communication with the first image-capture system and the second image-capture system and configured for:
      simultaneously capturing:
first video frames using the first image-capture system, the first video frames including the proximal portion; and
second video frames using the second image-capture system, the second video frames including the distal portion, an internal bone fiducial having an internal coordinate system, and an internal object;
   and
during the capturing:
for each of a plurality of pairs of the first video frames and the second video frames captured simultaneously:
processing the first video frame in the pair to determine a current external pose of the proximal portion in the external coordinate system;
processing the second video frame in the pair to:
determine a first current internal pose of the distal portion in the internal coordinate system;
determine a second current internal pose of the internal bone fiducial in the internal coordinate system; and
determine a third current internal pose of the internal object in the internal coordinate system;
calculating a pose of the internal object in the external coordinate system based on the current external pose, the first current internal pose, the second current internal pose, the third current internal pose, and the known pose;
   and
displaying, on a display device in association with the first video frames, indicia representing the pose of the internal object in the external coordinate system.
Clause 50. The surgical system of clause 49, wherein the second image-capture system comprises at least one of: an endoscopic camera and a needle scope.
Clause 51. The method of clause 49, wherein the first image-capture system comprises at least one of: a visual camera and an infrared camera.
Clause 52. The method of clause 49, where the visual camera and the display device are components of a head-mounted-display (HMD) device.
Clause 53. The method of clause 49, wherein the processing structure is configured for processing the first video frame to determine a current external pose of the proximal portion in the external coordinate system including:
processing the first video frame to determine the external pose of an external tool fiducial that is associated with the proximal portion.
Clause 54. The method of clause 49, wherein the processing structure is configured for processing the second video frame to determine a current internal pose of the distal portion in the internal coordinate system including:
processing the second video frame to determine the internal pose of an internal tool fiducial that is associated with the distal portion.

What is claimed is:

1. A method for registering internal and external coordinate systems of a surgical system, the method comprising:
providing a tool having a distal portion and a proximal portion;
capturing, while the proximal portion has a known pose with respect to the distal portion and while the distal portion is at a location within a surgical site and the proximal portion is outside of the surgical site, a pair of images comprising:
a first image using a first image-capture system and containing the proximal portion and an external bone fiducial; and
a second image using a second image-capture system and containing the distal portion and an internal bone fiducial;
processing the pair of images to:
determine an external pose of the proximal portion in an external coordinate system of the external bone fiducial; and
determine an internal pose of the distal portion in an internal coordinate system of the internal bone fiducial;
and
generating a spatial transformation between the external and internal coordinate systems based at least on the known pose.

2. The method of claim 1, further comprising:
capturing internal video frames of at least one object and the internal bone fiducial using the second image-capture system;
based on the spatial transformation, processing the internal video frames to determine a pose of the at least one object in the external coordinate system; and
displaying, on a display device in association with external video frames captured using the first image-capture system, indicia representing the pose of the at least one object in the external coordinate system.

3. The method of claim 1, further comprising:
capturing external video frames of at least one object and the external bone fiducial using the first image-capture system;
based on the spatial transformation, processing the external video frames to determine a pose of the at least one object in the internal coordinate system; and
displaying, on a display device in association with internal video frames captured using the second image-capture system, indicia representing the pose of the at least one object in the internal coordinate system.

4. The method of claim 1, further comprising:
forming the pair of images by simultaneously capturing the first image and the second image.

5. The method of claim 4, wherein the forming is conducted based at least on a known offset between a time of image capture by the first image-capture system and a time of image capture by the second image-capture system.

6. The method of claim 4, wherein the forming is conducted based at least on a known rate of image capture by the first image-capture system and a known rate of image-capture by the second image-capture system.

7. The method of claim 1, wherein the second image-capture system comprises at least one of: an endoscopic camera and a needle scope.

8. The method of claim 1, wherein the first image-capture system comprises at least one of: a visual camera and an infrared camera.

9. The method of claim 1, wherein the first image includes an external tool fiducial associated with the proximal portion, and wherein processing the pair of images to determine the external pose of the proximal portion in the external coordinate system of the external bone fiducial comprises:
processing the first image to determine the external pose of the external tool fiducial in the external coordinate system of the external bone fiducial.

10. The method of claim 1, wherein the second image includes an internal tool fiducial associated with the distal portion, and wherein processing the pair of images to determine the internal pose of the distal portion in the internal coordinate system of the internal bone fiducial comprises:
processing the second image to determine the internal pose of the internal tool fiducial in the internal coordinate system of the internal bone fiducial.

11. A surgical system comprising:
a tool comprising a distal portion and a proximal portion, the proximal portion having a known pose with respect to the distal portion, the distal portion dimensioned to be received within a surgical site while the proximal portion is outside of the surgical site;
a first image-capture system outside of the surgical site;
a second image-capture system inside the surgical site;
processing structure comprising at least one computer processor, the processing structure in communication with the first image-capture system and the second image-capture system and configured for:
capturing, while the distal portion is at a location within the surgical site and the proximal portion is outside of the surgical site, a pair of images comprising:
a first image using the first image-capture system and containing the proximal portion and an external bone fiducial; and
a second image using the second image-capture system and containing the distal portion and an internal bone fiducial;
processing the pair of images to:
determine an external pose of the proximal portion in an external coordinate system of the external bone fiducial; and
determine an internal pose of the distal portion in an internal coordinate system of the internal bone fiducial;
and
generating a spatial transformation between the external and internal coordinate systems based at least on the known pose.

12. The surgical system of claim 11, the processing structure configured for:
capturing internal video frames of at least one object and the internal bone fiducial using the second image-capture system;
based on the spatial transformation, processing the internal video frames to determine a pose of the at least one object in the external coordinate system; and
displaying, on a display device in association with external video frames captured using the first image-capture system, indicia representing the pose of the at least one object in the external coordinate system.

13. The surgical system of claim 11, the processing structure configured for:
capturing external video frames of at least one object and the external bone fiducial using the first image-capture system;
based on the spatial transformation, processing the external video frames to determine a pose of the at least one object in the internal coordinate system; and
displaying, on a display device in association with internal video frames captured using the second image-capture system, indicia representing the pose of the at least one object in the internal coordinate system.

14. The surgical system of claim 11, the processing structure configured for:
forming the pair of images by simultaneously capturing the first image and the second image.

15. The surgical system of claim 14, wherein the processing structure is configured for conducting the forming based at least on a known offset between a time of image capture by the first image-capture system and a time of image capture by the second image-capture system.

16. The surgical system of claim 14, wherein the processing structure is configured for conducting the forming based at least on a known rate of image capture by the first image-capture system and a known rate of image-capture by the second image-capture system.

17. The surgical system of claim 11, wherein the second image-capture system comprises at least one of: an endoscopic camera and a needle scope.

18. The surgical system of claim 11, wherein the first image-capture system comprises at least one of: a visual camera and an infrared camera.

19. The surgical system of claim 11, wherein the first image includes an external tool fiducial associated with the proximal portion, and wherein processing the pair of images to determine the external pose of the proximal portion in the external coordinate system of the external bone fiducial comprises:
processing the first image to determine the external pose of the external tool fiducial in the external coordinate system of the external bone fiducial.

20. The surgical system of claim 11, wherein the second image includes an internal tool fiducial associated with the distal portion, and wherein processing the pair of images to determine the internal pose of the distal portion in the internal coordinate system of the internal bone fiducial comprises:
processing the second image to determine the internal pose of the internal tool fiducial in the internal coordinate system of the internal bone fiducial.
